(19) United States
(12) Patent Application Publication
(10) Pub. No.: US 2025/0098644 A1
(43) Pub. Date: Mar. 27, 2025

(54) SYSTEMS AND METHODS FOR ANNOTATING PET HEALTH-RELATED SENSOR DATA

(71) Applicant: Mars, Incorporated, McLean, VA (US)

(72) Inventors: Nicholas KINLOCH, San Francisco, CA (US); David ALLEN, Mount Pleasant, SC (US); Aletha CARSON, Gaston, OR (US); Scott LYLE, San Francisco, CA (US); Maya BRUCK, Vancouver (CA); Nathanael YODER, San Francisco, CA (US); Robert CHAMBERS, Seattle, WA (US)

(21) Appl. No.: 18/890,218

(22) Filed: Sep. 19, 2024

Related U.S. Application Data
(60) Provisional application No. 63/584,290, filed on Sep. 21, 2023.

(51) Int. Cl. A01K 29/00

(52) U.S. Cl. CPC A01K 29/005

(57) ABSTRACT

A method for annotating pet health-related sensor data includes receiving from one or more sensors activity data indicative of one or more movements of a pet, determining a behavior of the pet based on the activity data, receiving contextual data associated with the pet from a user device, determining a temporal relationship between at least one data point of the behavior of the pet and at least one data point of the contextual data, determining a correlation between the behavior of the pet and at least one data point of the contextual data based on the temporal relationship, and providing a user interface for display on the user device. The user interface includes at least one graphic depicting the behavior annotated with the contextual data determined to be correlated with the behavior.

### CROSS-REFERENCE TO RELATED APPLICATION(S)

This patent application claims the benefit of priority to U.S. Application No. 63/584,290, filed on Sep. 21, 2023, the entirety of which is incorporated herein by reference.

### TECHNICAL FIELD

The present disclosure generally relates to methods and systems for annotating sensor data associated with pet activity, and more particularly, to annotating pet health-related sensor data with contextual data associated with the pet.

### BACKGROUND

Modern devices facilitate collection of a wide array of data relating to pet activity and behavior. For example, so-called “smart” collars use various sensors, such as accelerometers, to detect pet motion data. This raw data is then analyzed to determine specific behaviors performed by the pet. Knowledge of these behaviors can assist the pet owner or a care provider in monitoring changes in a pet's routine, or identifying the onset of a change in a pet's health or lifestyle.

However, motion data alone may be insufficient to conduct a holistic analysis of a pet's behavioral health. For example, raw sensor data acquired by sensors is not necessarily indicative of a real-world observation of the pet. More particularly, pet owners may have insights into their pet's emotional wellbeing and lifestyle changes that cannot be captured with sensor data exclusively.

This disclosure is directed to addressing above-referenced challenges. The background description provided herein is for the purpose of generally presenting the context of the disclosure. Unless otherwise indicated herein, the materials described in this section are not admitted to be prior art, or suggestions of the prior art, by inclusion in this section.

### SUMMARY OF THE DISCLOSURE

According to certain embodiments of the present disclosure, methods and systems are provided for annotating pet health-related sensor data. In some embodiments, a method for annotating pet health-related sensor data includes receiving, by at least one processor from one or more sensors, activity data indicative of one or more movements of a pet; determining, by the at least one processor, a behavior of the pet based on the activity data; receiving, by the at least one processor, contextual data associated with the pet from a user device; determining, by the at least one processor, a temporal relationship between at least one data point of the behavior of the pet and at least one data point of the contextual data; determining, by the at least one processor, a correlation between the behavior of the pet and at least one data point of the contextual data based on the temporal relationship; and providing, by the at least one processor, a user interface for display on the user device. The user interface includes at least one graphic depicting the behavior annotated with the contextual data determined to be correlated with the behavior.

Other embodiments are directed to a computer system for annotating pet health-related sensor data. The computer system includes at least one memory having processor-readable instructions stored therein, and at least one processor configured to access the at least one memory and execute the processor-readable instructions, which when executed by the at least one processor cause the at least one processor to perform a plurality of functions. The plurality of functions include functions for receiving, from one or more sensors, activity data indicative of one or more movements of a pet; determining a behavior of the pet based on the activity data; receiving contextual data associated with the pet from a user device; determining a temporal relationship between at least one data point of the behavior of the pet and at least one data point of the contextual data; determining a correlation between the behavior of the pet and at least one data point of the contextual data based on the temporal relationship; and providing a user interface for display on the user device. The user interface includes at least one graphic depicting the behavior annotated with the contextual data determined to be correlated with the behavior.

Other embodiments are directed to a non-transitory computer-readable medium configured to store instructions that, when executed by at least one processor of a device for annotating pet health-related sensor data, cause the at least one processor to perform operations. The operations include receiving, from one or more sensors, activity data indicative of one or more movements of a pet; determining a behavior of the pet based on the activity data; receiving contextual data associated with the pet from a user device; determining a temporal relationship between at least one data point of the behavior of the pet and at least one data point of the contextual data; determining a correlation between the behavior of the pet and at least one data point of the contextual data based on the temporal relationship; and providing a user interface for display on the user device. The user interface includes at least one graphic depicting the behavior annotated with the contextual data determined to be correlated with the behavior.

It is to be understood that both the foregoing general description and the following detailed description are exemplary and explanatory only and are not restrictive of the disclosed embodiments.

### BRIEF DESCRIPTION OF THE DRAWINGS

The accompanying drawings, which are incorporated in and constitute a part of this specification, illustrate various exemplary embodiments and together with the description, serve to explain the principles of the disclosed embodiments.

FIG. 1 depicts an exemplary platform environment, according to one or more embodiments.

FIG. 2 depicts an exemplary environment of a pet owner profile and corresponding pet profiles, according to one or more embodiments.

FIG. 3A depicts a flowchart of an exemplary method, according to one or more embodiments.

FIG. 3B depicts a flowchart of another exemplary method, according to one or more embodiments.

FIG. 3C depicts a flowchart of a further exemplary method, according to one or more embodiments.

FIGS. 4A-4D depict exemplary user interfaces displayed on a user device when a change in a pet behavior is determined, according to one or more embodiments.

FIG. 5 depicts an exemplary user interface including a time-series plot of pet behavior displayed on a user device, according to one or more embodiments.

FIGS. 6A-6D depict exemplary user interfaces displayed on a user device when a change in a pet behavior is determined, according to one or more embodiments.

FIGS. 7A-7B depict an exemplary user interface including graphs of pet behavior displayed on a user device, according to one or more embodiments.

FIGS. 8A-8B depict exemplary user interfaces displayed on a user device for facilitating input of pet mood data, according to one or more embodiments.

FIGS. 9A-9G depict exemplary user interfaces displayed on a user device for facilitating input of pet mood analysis and digital journaling of pet mood data, according to one or more embodiments.

FIG. 10 depicts an exemplary environment that may be utilized with the techniques presented hereon, according to one or more embodiments.

FIG. 11 depicts an exemplary computing device that may execute the techniques described herein, according to one or more embodiments.

### DETAILED DESCRIPTION OF EMBODIMENTS

According to certain aspects of the disclosure, methods and systems are disclosed for correlating pet health-related sensor data to pet contextual data. In some embodiments, the methods and system described herein may be used to overlay and/or annotate time-series sensor data indicative of pet movements and/or behavior with pet contextual data received from the pet's owner.

Sensor data may be obtained from devices such as “smart” collars which utilize various sensors, such as accelerometers, to collect data relating to pet motion. This raw motion data may then be processed and/or analyzed to identify behaviors, such as scratching, licking, walking, lying down, eating, drinking, etc., performed by the pet. However, this data often lacks context (e.g., does not capture a comprehensive view) of the overall life of the pet. As such, collection and analysis of additional, contextual data to supplement the sensor data may be desirable to make more robust and comprehensive pet health-related determinations and/or recommendations, for example.

Accordingly, there exists a need for methods and systems for correlating sensor data obtained from devices, such as smart collars, with contextual data provided from other sources. The methods and systems of the present disclosure address this need by receiving data from a pet owner (or other source) and combining that data with sensor data in various manners as described herein. The data received from the pet owner (or other sources) may include pet contextual data related to the lifestyle of the pet (e.g. changes to food or medication) and/or data related to the pet's emotional health that supplements and/or provides context to the sensor data.

The terminology used below may be interpreted in its broadest reasonable manner, even though it is being used in conjunction with a detailed description of certain specific examples of the present disclosure. Indeed, certain terms may even be emphasized below; however, any terminology intended to be interpreted in any restricted manner will be overtly and specifically defined as such in this Detailed Description section. Both the foregoing general description and the following detailed description are exemplary and explanatory only and are not restrictive of the features.

In this disclosure, the term “based on” means “based at least in part on.” The singular forms “a,” “an,” and “the” include plural referents unless the context dictates otherwise. The term “exemplary” is used in the sense of “example” rather than “ideal.” The terms “comprises,” “comprising,” “includes,” “including,” or other variations thereof, are intended to cover a non-exclusive inclusion such that a process, method, or product that comprises a list of elements does not necessarily include only those elements, but may include other elements not expressly listed or inherent to such a process, method, article, or apparatus. The term “or” is used disjunctively, such that “at least one of A or B” includes, (A), (B), (A and A), (A and B), etc. Relative terms, such as, “substantially” and “generally,” are used to indicate a possible variation of ±10% of a stated or understood value.

As used herein, a term such as “user” or the like generally encompasses a future pet owner, future pet owners, pet owner, and/or pet owners. A term such as “pet” or the like generally encompasses a domestic animal, such as a domestic canine, feline, rabbit, ferret, horse, cow, or the like. In exemplary embodiments, “pet” may refer to a canine.

As used herein, the term “structured data” refers to data stored in a predefined format which can be directly compared with other data of the same format, and/or otherwise used without further processing. For example, structured data may include data generated based on a user's selection of one of a predefined list of options. As used herein, the term “unstructured data” refers to data which is stored in a native format not necessarily directly comparable with other data. For example, unstructured data may include data generated based on a user's input into a text field. Unstructured data may need to be processed into a predefined format, such as by extracting pertinent information from free-from text, in order to make use of the data.

### Exemplary Platform Environment

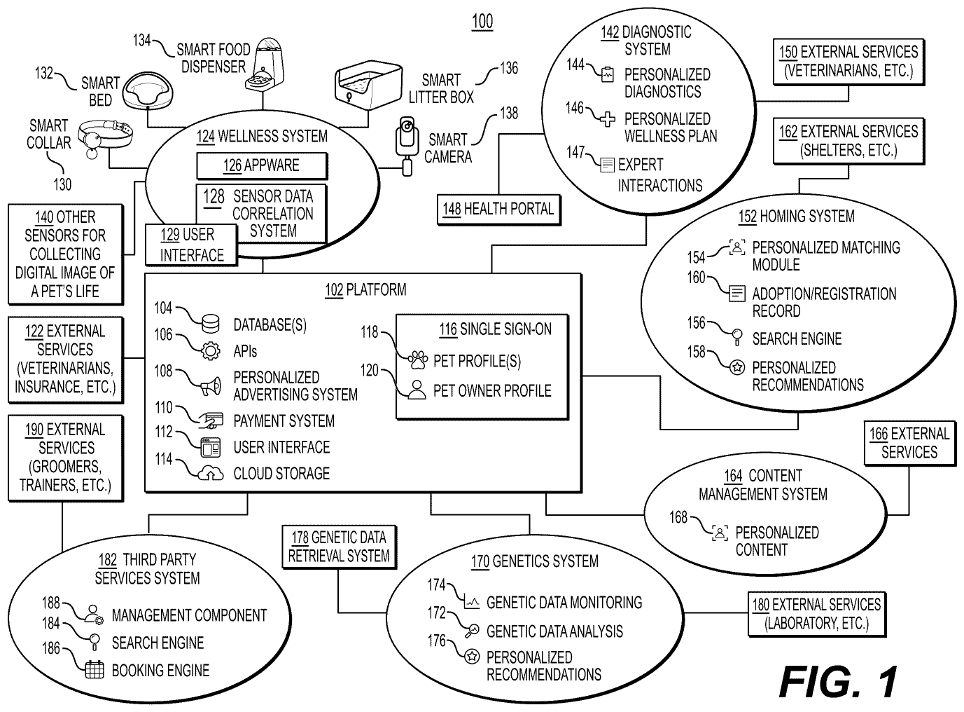

FIG. 1 depicts an exemplary platform environment 100 that may be utilized with the techniques presented herein. More specifically, environment 100 may provide an integrated hardware and software platform 102 for improving pet digitalization by centralizing the pet's information.

The platform 102 may communicate with one or more external systems that may collect, manage, and store different types of pet data and/or pet owner data. The platform 102 may retrieve the pet data and/or pet owner data from the one or more external systems via APIs 106. In some embodiments, the platform 102 may store the pet data and/or the pet owner data. For example, the platform 102 may store the pet data in pet profile(s) 118. Additionally, for example, the platform 102 may store the pet owner data in a pet owner profile 120. The one or more external systems may include at least one of a wellness system 124, a diagnostic system 142, a homing system 152, a content management system 164, a genetics system 170, and/or a third party services system 182. Such external systems are described in more detail below.

The platform 102 may also communicate with one or more external services. In some embodiments, the platform 102 may communicate with the one or more external services via APIs 106. External services 122 may include, for example, one or more third party and/or auxiliary systems that integrate and/or communicate with the platform 102 in performing various pet tasks. For example, the external services 122 may include at least one of: a veterinarian, a pet behaviorist, a pet nutritionist, a pet insurance agency, a pet service provider, and the like.

The platform 102 may include database(s) 104 and/or cloud storage 114 that may store information corresponding to one or more pets and/or one or more pet owners. For example, the database(s) 104 and/or cloud storage 114 may store the pet profile(s) 118 and/or the pet owner profile 120. The database(s) 104 and/or the cloud storage 114 may be located internally or externally to platform 102.

The platform 102 may include a personalized advertising system 108 and/or a payment system 110. The personalized advertising system 108 may create and/or display personalized advertisements to the user. For example, the personalized advertisements may be created based on information contained the in pet profile(s) 118 and/or the pet owner profile 120. In some embodiments, the personalized advertising system 108 may display the personalized advertisements on a user interface 112 of the platform 102. The payment system 110 may allow the user to create a financial account for a pet and/or perform financial transactions for pet services and/or pet goods (e.g., using pet owner digital wallet 216).

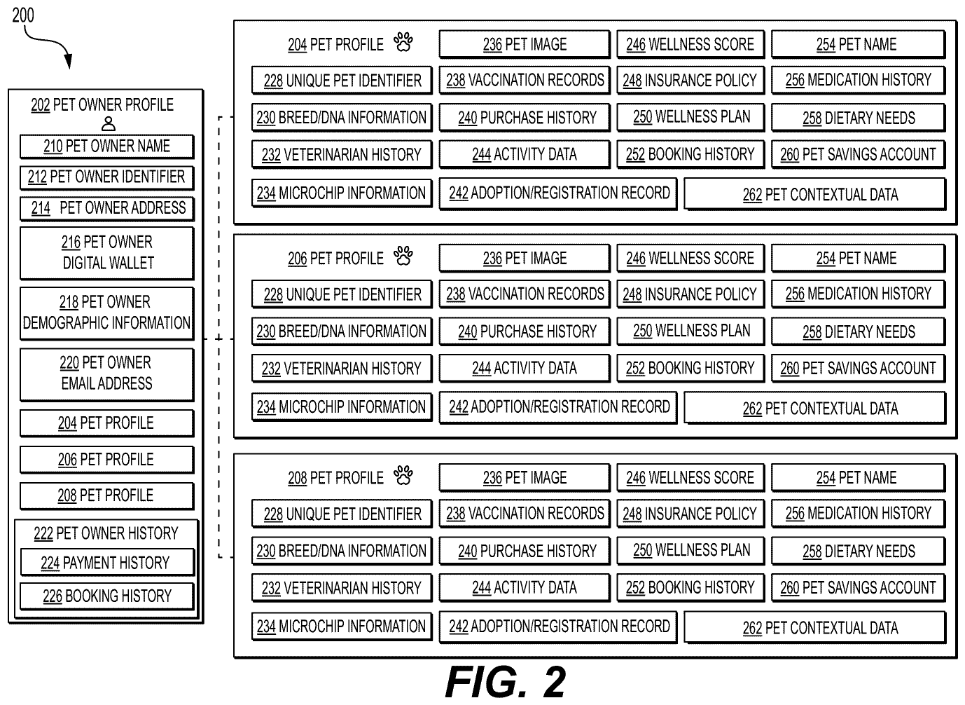

The platform 102 may include a single sign-on 116. The single sign-on 116 may include a unique identifier that may correspond to the pet profile(s) 118 and/or the pet owner profile 120. Each of the pet profile(s) 118 may include information corresponding to a particular pet. The pet owner profile 120 may include information corresponding to a particular pet owner. Additionally, the pet owner profile 120 and/or the pet profile(s) 118 may each have a corresponding avatar and/or virtual presence. The avatar and/or virtual presence may include different attributes that are shared by the pet owner and/or pets. The pet profile(s) 118 and the pet owner profile 120 are described in further detail in the description of FIG. 2.

### Wellness System

The wellness system 124 may collect, manage, and/or display wellness data of a pet. The wellness system 124 may be an internal component or an external component of the platform 102, where the wellness system 124 may communicate with the platform 102 via APIs 106.

The wellness system 124 may collect data from one or more smart devices. The wellness system 124 may communicate with the one or more smart devices via one or more APIs. Additionally, in some embodiments, the wellness system 124 may use appware 126 to facilitate the communication and/or the management of the one or more smart devices. For example, the appware 126 may communicate with one or more smart devices that may run on an external system. Additionally, for example, the appware 126 may run on a user device, where the appware 126 provides a user interface 129 to display the data collected by the one or more smart devices. In some embodiments, the appware 126 may manage one or more smart devices. The wellness system 124 may communicate with the one or more smart devices by sending one or more requests to the one or more smart devices. The requests may ask the one or more smart devices to send collected wellness data to the wellness system 124. In some embodiments, the one or more smart devices may automatically send wellness data to the wellness system 124. For example, the one or more smart devices may send the wellness data to the wellness system 124 at regular time intervals (e.g., every 30 seconds, every hour, every day, and the like) and/or whenever new wellness data is collected. In some embodiments, the wellness system 124 may store the wellness data in an internal or external storage. For example, the wellness system 124 may store the wellness data in the database(s) 104 and/or the cloud storage 114. Additionally, or alternatively, for example, the wellness system 124 may store the wellness data in the pet profile(s) 118 and/or the pet owner profile 120.

The wellness system 124 further includes a sensor data correlation system 128 configured to correlate data received from the smart devices with data received from a user device via the user interface 129. In particular, the sensor data correlation system 128 may annotate data gathered, collected, and/or received from the smart devices with contextual data received via user interface 129, based on temporal relationships of the data. In some embodiments, the sensor data correlation system 128 may be configured to generate prompts, graphics, control elements, input fields and the like for display via the user interface 129. In some embodiments, the sensor data correlation system 128 may be configured to predict a potential cause of a change in the pet's behavior based on a correlation between the data received from the smart devices with data received from a user device. In some embodiments, the sensor data correlation system 128 may utilize one or more machine learning models to make the prediction of the potential cause of a change in the pet's behavior.

In some examples, the wellness system 124 may include one or more additional systems configured to analyze data received from the smart devices. As one example, the wellness system 124 may include a wellness index scoring system configured to analyze the data received from the smart devices to determine a wellness score for output via the user interface 129.

Example smart devices may include at least one of: a smart collar 130, a smart bed 132, a smart food dispenser 134, a smart litter box 136, a smart camera 138, and/or any other sensors 140 for collecting activity and/or behavioral data of a pet.

The smart collar 130 may include a device and/or a sensor that may attach to a pet. For example, the smart collar 130 may attach around the pet's neck. The smart collar 130 may detect a pet's activity, location, and eating information, such as physical activity, location, and the like using one or more sensors of the smart collar 130. Example sensors may include accelerometers, thermometers, gyroscopes, altimeters, etc. Specific examples of physical activity that may be detected by the smart collar 130 include scratching, licking, walking, lying down, sleeping, eating, drinking, and the like. The smart collar 130 may collect the activity, location, and eating information of the pet and send such information to the wellness system 124. In some embodiments, the smart collar 130 may automatically send the activity, location, and eating information to the wellness system 124 after a set period of time. In some embodiments, the smart collar 130 may send the activity, location, and eating information in response to a request from the wellness system 124. Such a request may be conveyed to the owner of the pet, for example, via the user interface 129.

The smart bed 132 may include a device and/or a sensor that may be included in a pet bed. The smart bed 132 may track sleeping information corresponding to the pet. The sleeping information may include the amount of time a pet sleeps in the smart bed 132, how frequently the pet gets up from the smart bed 132, if the pet tosses and turns while sleeping, and the like. The smart bed 132 may send such information to the wellness system 124. In some embodiments, the smart bed 132 may automatically send the sleeping information to the wellness system 124 after a set period of time. In some embodiments, the smart bed 132 may send the sleeping information in response to a request from the wellness system 124.

The smart food dispenser 134 may include a device and/or a sensor that may be included in a pet food feeder. The smart food dispenser 134 may track how much food is dispensed for the pet to eat. The smart food dispenser 134 may send such food dispensing information to the wellness system 124. In some embodiments, the smart food dispenser 134 may automatically send the food dispensing information to the wellness system 124 after a set period of time. In some embodiments, the smart food dispenser 134 may send the food dispensing information in response to a request from the wellness system 124.

The smart litter box 136 may include a device and/or a sensor that may be included in a litter box. The smart litter box 136 may track a pet's litter box information. The litter box information may include at least one of: how frequently the pet uses the smart litter box 136, what the pet does in the smart litter box 136, and the like. In some embodiments, the smart litter box 136 may automatically send the litter box information to the wellness system 124. In some embodiments, the smart litter box 136 may automatically send the litter box information to the wellness system 124 after a set period of time. In some embodiments, the smart litter box 136 may send the litter box information in response to a request from the wellness system 124.

The smart camera 138 may include a device and/or a sensor that may be included in a camera. The smart camera 138 may capture behavior information of a pet. The pet's behavior information may include physical activity, eating food from the pet's food dish, eating food from a source different from the pet's food dish, drinking from the pet's drinking dish, drinking from a source different from the pet's drinking dish, and the like. In some embodiments, the smart camera 138 may automatically send the behavior information to the wellness system 124 after a set period of time. In some embodiments, the smart camera 138 may send the behavior information in response to a request from the wellness system 124. Such a request may be conveyed to the owner of the pet, for example, via the user interface 129.

The other sensors 140 for collecting activity and/or behavioral data of the pet may include one or more devices and/or one or more sensors that collect data relating to the pet's activity, location, and eating information, such as physical activity, location, and the like. Specific examples of physical activity that may be detected by the smart collar 130 include scratching, licking, walking, lying down, sleeping, eating, drinking, and the like. In some embodiments, the other sensors 140 may automatically send the collected data to the wellness system 124 after a set period of time. In some embodiments, the other sensors 140 may send the collected data in response to a request from the wellness system 124. Such a request may be conveyed to the owner of the pet, for example, via the user interface 129.

### Diagnostic System

The diagnostic system 142 may manage a pet's health information and provide personalized diagnostics 144 and/or a personalized wellness plan 146 to the user. Additionally, the diagnostic system 142 may facilitate interactions between pet experts and pet owners, hereinafter referred to as expert interactions 147. The diagnostic system 142 may be an internal component or an external component of the platform 102, where the diagnostic system 142 may communicate with the platform 102 via APIs 106.

The diagnostic system 142 may manage a pet's heath information (e.g., vaccination records, medical records) by receiving the pet's health information from one or more external services 150 (e.g., veterinarians, clinics, pet hospital, interactive chats, and the like). The diagnostic system 142 may store the pet's health information in the pet profile(s) 118. In an embodiment, the diagnostic system 142 may communicate the pet's health information using APIs 106.

The diagnostic system 142 may create personalized diagnostics 144 and/or a personalized wellness plan 146 based on the pet's health information. The personalized diagnostics 144 may include one or more diagnoses (e.g., ear infection, eye infection, and the like) of medical conditions for the pet. In some embodiments, the personalized diagnostics 144 may be based on diagnoses and/or observations made by the external services 150. In some embodiments, the personalized diagnostics 144 may be based on diagnoses and/or observation determined by the sensor data correlation system 128 of the wellness system 124 utilizing one or more machine learning models. The personalized wellness plan 146 may include one or more recommendations regarding eating events, exercise events, health checks and wellness visits, and the like, which may be based on the pet's heath information. The personalized wellness plan 146 may be based on recommendations made by the external services 150. The personalized wellness plan 146 may be based on information included in the pet profile(s) 118. In some embodiments, the personalized wellness plan 146 may be based on one or more recommendations made by one or more machine learning models.

The diagnostic system 142 may facilitate the expert interactions 147 during which a pet owner interacts with an expert in various fields, such a as veterinarian, a veterinary technician, a pet behaviorist, a pet nutritionist, or the like. The expert may be associated with one of the external services 150, and may provide remote, online, and/or virtual services to conduct appointments, provide consultations, etc. with the pet and pet owner. Interaction with the expert may include, for example, an audio call, a video call, a text-based chat, or the like. In some embodiments, the expert interactions 147 may be initiated via the pet owner's interaction with the user interface 129 displayed on a user device via the appware 126.

The health portal 148 may provide access to one or more parties who wish to retrieve the personalized diagnostics 144, personalized wellness plan 146, expert interactions 147, and/or the pet's health information from the pet profile(s) 118. The health portal 148 may be internal or external to the diagnostic system 142. Additionally, the health portal 148 may include a user interface. For example, a groomer may access the health portal 148 to retrieve the pet's vaccination records from diagnostic system 142.

The diagnostic system 142 may communicate with one or more of the external services 150, such as veterinarians, clinics, pet hospital, virtual experts, and the like. For example, one of the external services 150 (e.g., veterinarian) may send updated vaccine or medical records to the diagnostic system 142, where the diagnostic system 142 may then store such updated vaccine or medical records in the pet profile(s) 118. Additionally, for example, the diagnostic system 142 may update the personalized diagnostics 144 and/or the personalized wellness plan 146 based on the updated vaccine or medical records. In the case of virtual experts, the external services 150 may send data generated during remote, virtual, and/or online interactions with the virtual experts to diagnostic system 142. The diagnostic system 142 may then store such data, in the form of one or more of a transcript, an audio recording, a video recording, one or more images, and/or notes in the pet profile(s) 118.

In some embodiments, the diagnostic system 142 may include information to authenticate the pet. For example, social media websites frequently require that a user is authenticated in order to label the user as “verified” (e.g., a blue checkmark). The diagnostic system 142 may contain information corresponding to a physical examination of the pet. Such information may include authentication information of the pet. For example, the authentication information may include a confirmation of the pet's breed, gender, image, etc. Such authentication information may be used by a social media website to authenticate the pet as a “verified” user.

### Homing System

The homing system 152 may match a future pet owner with a pet and provide additional support for the future pet owner. The homing system 152 may be an internal component or an external component of the platform 102, where the homing system 152 may communicate with the platform 102 via APIs 106.

The homing system 152 may match a future pet owner with a particular pet using a personalized matching module 154 and/or a search engine 156. The personalized matching module 154 may use user information (e.g., user location, user age, and the like) from the future pet owner (e.g., from the pet owner profile 120) to automatically search for one or more pets that are best suited for the future pet owner. In some embodiments, the personalized matching module 154 may use one or more machine learning models to determine the best pet matches for the future pet owner. The search engine 156 may allow the future pet owner to search for one or more pets. The search engine 156 may include different search filters (e.g., filtering by breed, age, size, weight, and the like), which may allow the user to filter the results of the one or more pets.

Both the personalized matching module 154 and/or the search engine 156 may retrieve results from one or more external services 162. The external services 162 may include one or more of: a pet adoption agency, a shelter, a pet breeder, and the like. When the personalized matching module 154 and/or the search engine 156 is performing a search for one or more pets, the personalized matching module 154 and/or the search engine 156 may send one or more requests to the external services 162 for available pets that fit one or more parameters contained in the one or more requests. Upon receiving the one or more requests, the external services 162 may search one or more databases for one or more matching pets. The external services 162 may send a response to the personalized matching module 154 and/or the search engine 156. The response may include the one or more matching pets. Alternatively, for example, if no matching pets were found, the response may include an indicator that no matching pets were found. In some embodiments, the homing system 152 may store the one or more matching pets in a database, such as an internal database or an external database (e.g., one of the database(s) 104).

The homing system 152 may display the one or more matching pets to the future pet owner, along with an option for the future pet owner to adopt and/or purchase the one or more matching pets. The homing system 152 may also facilitate the adoption and/or purchase of the one or more matching pets. In some embodiments, the homing system 152 may communicate with the external services 162 to facilitate the adoption and/or purchase of the one or more matching pets.

Once the future pet owner purchases and/or adopts the pet, the homing system 152 may store and/or manage the pet's adoption/registration record 160. In some embodiments, the homing system 152 may receive all (or part of) the pet's adoption/registration record 160 from the external services 162. In some embodiments, the homing system 152 may store the pet's adoption/registration record 160 in the pet profile(s) 118. Additionally, or alternatively, the homing system 152 may store the pet's adoption/registration record in the pet owner profile 120. In some embodiments, the homing system 152 may store the pet's adoption/registration record 160 in an internal or external database (e.g., one of the database(s) 104).

The homing system 152 may provide additional support for the future pet owner by providing personalized recommendations 158 to the pet owner. The personalized recommendations 158 may be based on characteristics of the pet that the future pet owner purchased and/or adopted. Example personalized recommendations 158 may include a recommended pet food, a recommended pet provider, recommended pet supplies, and the like. In some embodiments, the personalized recommendations 158 may be based on communications with one or more of the external services 162 and/or other systems associated with the platform 102. For example, the homing system 152 may communicate with the content management system 164 to receive personalized content 168, and then make personalized recommendations 158 based on the personalized content 168.

### Content Management System

The content management system 164 may provide personalized content 168 to a user. The content management system 164 may be an internal component or an external component of platform 102, where the content management system 164 may communicate with the platform 102 via APIs 106.

The content management system 164 may retrieve the personalized content 168 and display such personalized content 168 to the user. The personalized content 168 may include at least one of: an article, a blog post, an online forum, an advertisement, and the like. The personalized content 168 may also include recommendations that are specific towards the pet and/or user. The recommendations may include food recommendations, activity recommendations, product recommendations, resource recommendations (e.g., books, articles, and the like), third party services recommendations (e.g., groomer, trainer, boarding), and the like. The personalized content 168 may be personalized based on the pet profile(s) 118 and/or the pet owner profile 120. The content management system 164 may display the personalized content 168 via a user interface of a user device. In some embodiments, the content management system 164 may retrieve the personalized content 168 from one or more external services 166. The external services 166 may include an electronic magazine, one or more databases, one or more social media posts, and the like. In some embodiments, the content management system 164 may retrieve the personalized content 168 from other sources, such as the database(s) 104, the cloud storage 114, and the personalized advertising system 108. In some embodiments, the content management system 164 may create personalized content 168 based on communications with the other external systems (e.g., wellness system 124, diagnostic system 142, homing system 152, genetics system 170, third party services system 182, etc.). For example, the content management system 164 may receive the personalized wellness plan 146 from diagnostic system 142. The personalized content 168 may then be based on (or include) information from the personalized wellness plan 146. In some embodiments, the personalized content 168 may be based on correlations determined by the sensor data correlation system 128 of the wellness system 124.

### Genetics System

The genetics system 170 may analyze and/or monitor a pet's genetic data. The genetics system 170 may be an internal component or an external component of the platform 102, where the genetics system 170 may communicate with the platform 102 via APIs 106.

The genetics system 170 may include genetic data analysis 172, genetic data monitoring 174, and/or personalized recommendations 176. Additionally, the genetic data analysis 172 and/or the genetic data monitoring 174 may communicate with one or more external services 180 to assist with the analysis and/or the monitoring of the genetic data. The external services 180 may include a laboratory, a clinic, a veterinarian, and the like.

The genetic data analysis 172 may receive genetic data belonging to a pet. In some embodiments, the genetic data analysis 172 may receive the genetic data from a genetic data retrieval system 178. The genetic data retrieval system 178 may retrieve and store genetic data belonging to one or more pets. Additionally, the genetic data analysis may receive genetic data from the genetic data retrieval system 178, where the received genetic data is used in the analysis of the genetic data belonging to the pet. The genetic data analysis 172 may analyze the genetic data to determine abnormalities, potential genetic traits, familial relationships, and the like. In some embodiments, the genetic data analysis 172 may communicate with one or more of the external services 180 to assist with the analysis of the genetic data. For example, the genetic data analysis 172 may send genetic data information to a laboratory for the laboratory to perform the analysis of the genetic data.

The genetic data monitoring 174 may monitor the genetic data belonging to a pet to determine any changes in the genetic data. For example the genetic data monitoring 174 may receive new genetic data and compare the new genetic data to previously stored genetic data. The comparing may lead the genetic data monitoring 174 to determine that there is an abnormality or an improvement in the genetic data. In some embodiments, the genetic data monitoring 174 may communicate with one or more of the external services 180, in order for the external services 180 to analyze the genetic data and determine if there are any changes.

The genetics system 170 may provide personalized recommendations 176 to the user. For example, the genetics system 170 may provide personalized recommendations 176 to the user via a user interface of a user device. For example, the personalized recommendations 176 and/or genetic-related data determined by the genetics system 170 may be displayed on the user interface 129 associated with the wellness system 124 via the appware 126. In some embodiments, the personalized recommendations 176 may be based on the genetic data analysis 172 and/or the genetic data monitoring 174. The personalized recommendations 176 may include a pet food recommendation, an exercise recommendation, a pet item recommendation, health checks or wellness visits, and the like.

In some embodiments, the personalized recommendations 176 may be based on communications with one or more of the external services 180 and/or other systems associated with the platform 102. For example, the genetics system 170 may communicate with the diagnostic system 142. The genetics system 170 may send a request to the diagnostic system 142 for a personalized wellness plan 146. The request may include, for example, the genetic data analysis 172 and/or the genetic data monitoring 174. The diagnostic system 142 may communicate the personalized wellness plan 146 to the genetics system 170, where the personalized wellness plan 146 may be based on the genetic data analysis 172 and/or the genetic data monitoring 174. The genetics system 170 may make personalized recommendations 176 to the user based on the personalized wellness plan 146.

In some embodiments, the genetics system 170 may provide the genetic data analysis 172 and/or the genetic data monitoring 174 to the sensor data correlation system 128 of the wellness system 124 to contextualize pet activity detected by the smart collar 130, smart camera 138, etc. That is, the sensor data correlation system 128 may utilize the genetic data analysis 172 and/or the genetic data monitoring 174 to inform pet health-related predictions and/or diagnoses.

In some embodiments, the genetics system 170 may include information to authenticate the pet. For example, social media websites frequently require that a user is authenticated in order to label the user as “verified” (e.g., a blue checkmark). The genetics system 170 may contain information corresponding to a physical examination of the pet. Such information may include authentication information of the pet. For example, the authentication information may include a confirmation of the pet's breed, gender, image, etc. Such authentication information may be used by a social media website to authenticate the pet as a “verified” user.

### Third Party Services System

The third party services system 182 may allow a user to search for and reserve different external services 190, such as groomers, trainers, veterinarians, sitters, holistic care (e.g., nutritionist, naturopathic), and the like. The third party services system 182 may be an internal component or an external component of the platform 102, where the third party services system 182 may communicate with the platform 102 via APIs 106.

The third party services system 182 may include a search engine 184, a booking engine 186, and/or a management component 188.

The search engine 184 may allow the user, such as a pet owner, to search for one or more of the external services 190 to reserve for the user's pet. The search engine 184 may include filtering functionality to facilitate a fine-tuned search. The filtering functionality may include universal filtering and/or service specific filtering. For example, the universal filtering may include filtering the external services 190 by location, price range, and/or ratings. Additionally, for example, the service specific filtering may include filtering the external services 190 by breed specialty, health issues, and/or behavioral needs.

The booking engine 186 may allow the user to reserve the external services 190. For example, after using the search engine 184 to search for external services 190, the user may use the booking engine 186 to reserve a particular service of the external services 190. The booking engine 186 may present open dates and time slots, which may correspond to the selected external service 190. The user may then use the booking engine 186 to select a date and/or time from the displayed open dates and time slots. Upon the finalization of the booking, the user may receive an instant confirmation of the booking, such as via text or email. The user may also have the ability to instantly pay for the booked service. Alternatively, the user may be able to pay upon the finalization of the service. The user may be able to upload photos and include notes to the external service 190. For example, the user may upload dog photos to a groomer, or make a note that the user's dog has a limp.

The management component 188 may provide functionality to manage the different external services 190. For example, the management component 188 may provide the functionality for the external services 190 to register and/or be removed from the third party services system 182. The management component 188 may communicate with one or more databases (e.g., the database(s) 104) and/or cloud storage (e.g., the cloud storage 114) to store information (e.g., a name, a business identifier, a specialty, and the like) corresponding to the external services 190.

### Exemplary Pet Owner Profile and Pet Profile(s)

FIG. 2 depicts an exemplary environment 200 of a pet owner profile 202 and corresponding pet profiles that may be utilized with the techniques presented herein. Notably, exemplary environment 200 may complement exemplary platform environment 100, with a pet owner profile 202 corresponding to the pet owner profile 120 of FIG. 1. Additionally, a pet profile 204, a pet profile 206, and/or a pet profile 208 may correspond to the pet profile(s) 118 of FIG. 1.

The pet owner profile 202 may include at least one of: a pet owner name 210, a pet owner identifier 212, a pet owner address 214, a pet owner digital wallet 216, pet owner demographic information 218, a pet owner email address 220, at least one pet profile (e.g., the pet profile 204, the pet profile 206, the pet profile 208) and/or at least one identifier associated with the at least one pet profile, and/or a pet owner history 222. The pet owner name 210 may include a name of the pet owner. The pet owner identifier 212 may include a unique identifier that may be used to locate the pet owner profile 202. In some embodiments, the pet owner identifier 212 may allow for tracking of some or all of the user's activities. The pet owner address 214 may include a physical address of the pet owner. The pet owner digital wallet 216 may include payment information, such as credit card information, cryptocurrency information, and the like. The pet owner demographic information 218 may include a particular demographic of the pet owner. The pet owner email address 220 may include an email address of the pet owner. The pet owner profile may include at least one pet profile (e.g., the pet profile 204, the pet profile 206, and/or the pet profile 208). In some embodiments, in lieu of including an entirety of the at least one pet profile, the pet owner profile 202 may include at least one identifier associated with the at least one pet profile (e.g., a unique pet identifier 228). Each of the pet profiles may correspond to a pet that belongs to the pet owner. The number of pet profiles may be dynamic, where the pet profiles may adjust according to the number of pets that belong to the user.

The pet owner history 222 may include a payment history 224 and/or a booking history 226. The payment history 224 may include financial transactions of the pet owner. In some embodiments, the payment history 224 may correspond to activity of the pet owner digital wallet 216. In some embodiments, the payment history 224 may be tracked and analyzed to provide for targeted advertising (e.g., of personalized advertising system 108) and/or recommendations to the pet owner. The booking history 226 may include previous bookings of third party services that were made by the user. In some embodiments, the booking history 226 may be tracked and analyzed to provide for targeted advertising (e.g., of personalized advertising system 108) and/or recommendations to the pet owner.

The pet profiles 204, 206, and/or 208 may each correspond to a different pet that belongs to the pet owner of the pet owner profile 202. The pet owner may have more or less than three pets. The number of pet profiles may be dynamic, where the number of pet profiles corresponds to the number of pets that belong to the pet owner. In some embodiments, the pet owner may want only a subset of the pet owner's pets to have pet profiles.

The pet profiles 204, 206, and/or 208 may each include at least one of: a unique pet identifier 228, breed/DNA information 230, veterinarian history 232, microchip information 234, a pet image 236, vaccination records 238, a purchase history 240, an adoption/registration record 242, activity data 244, a wellness score 246, an insurance policy 248, a wellness plan 250, a booking history 252, a pet name 254, medication history 256, dietary needs 258, a pet savings account 260, and/or pet contextual data 262.

The unique pet identifier 228 may include a unique identifier that may be used to locate the corresponding pet profile (e.g., the pet profiles 204, 206, and/or 208). In some embodiments, the unique pet identifier 228 may allow for tracking of some or all of activities corresponding to the pet.

The pet image 236 may include a photograph, drawing, virtual presence, and/or avatar of the pet. The pet name 254 may include the name of the pet and/or any nicknames. The insurance policy 248 may include a pet insurance policy for the pet. The purchase history 240 may include purchases made for the pet. The pet savings account 260 may include a financial savings account for the pet. In some embodiments, the pet image 236, the pet name 254, the purchase history 240, pet savings account 260, and/or the insurance policy 248 may have been received from one or more of the external systems.

The breed/DNA information 230 may correspond to the breed and/or DNA information of the pet. In some embodiments, the breed/DNA information 230 may have been received from one or more of the external systems. For example, the breed/DNA information 230 may have been received from genetics system 170.

The veterinarian history 232 may include the details of the pet's visit(s) to a veterinarian. The veterinarian history 232 may also include notes from the vet and/or possible diagnoses and treatments. The vaccination records 238 may include one or more vaccination records of vaccinations administered to the pet. The medication history 256 may include details of the medications that the pet currently takes and/or has taken in the past. The dietary needs 258 may include information regarding food that the pet should eat and/or food that the pet should avoid. The wellness plan 250 may correspond to a wellness plan for the pet. In some embodiments, the wellness plan 250 may have been determined based on the personalized wellness plan 146. In some embodiments, the veterinarian history 232, vaccination records 238, dietary needs 258, wellness plan 250, and/or the medication history 256 may have been received from one or more of the external systems. For example, the veterinarian history 232, vaccination records 238, dietary needs 258, wellness plan 250, and/or the medication history 256 may have been received from diagnostic system 142.

The microchip information 234 may include a microchip number of the pet. For example, the microchip may have been inserted into the pet to track the pet. The adoption/registration record 242 may include documentation of the adoption or purchase of the pet. In some embodiments, the microchip information 234 and/or adoption/registration record 242 may have been received from one or more of the external systems. For example, the microchip information 234 and/or adoption/registration record 242 may have been received from homing system 152.

The activity data 244 may include data indicative of one or more movements or behaviors of the pet. For example, the activity data 244 may be collected by a smart collar 130, a smart bed 132, a smart food dispenser 134, a smart litter box 136, a smart camera 138, and/or the other sensors 140 for collecting activity and/or behavioral data of a pet's life. The wellness score 246 may include data corresponding to a wellness score produced by a sub-system of the wellness system 124, such as the wellness index scoring system. In some embodiments, the activity data 244 and/or the wellness score 246 may have been received from one or more of the external systems. For example, the activity data 244 and/or the wellness score 246 may have been received from the wellness system 124.

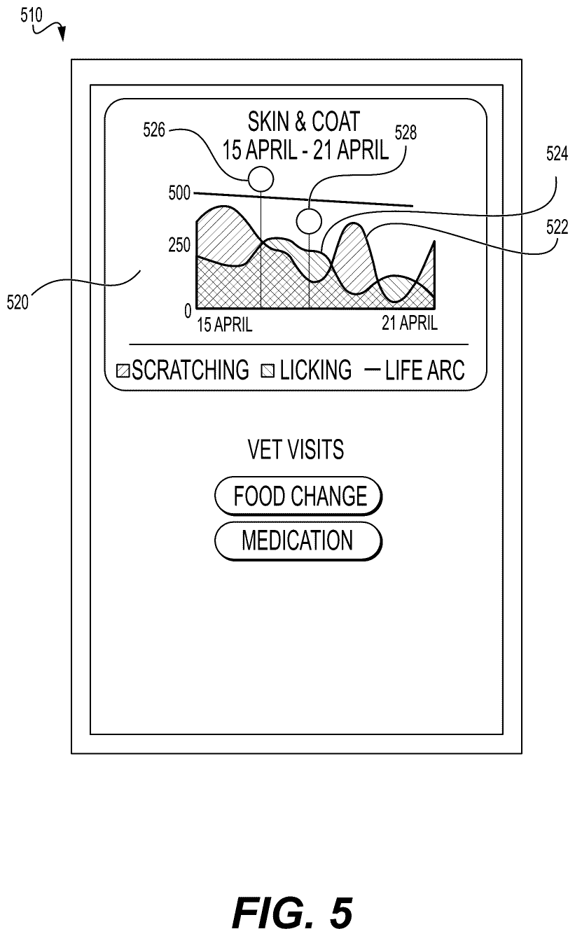

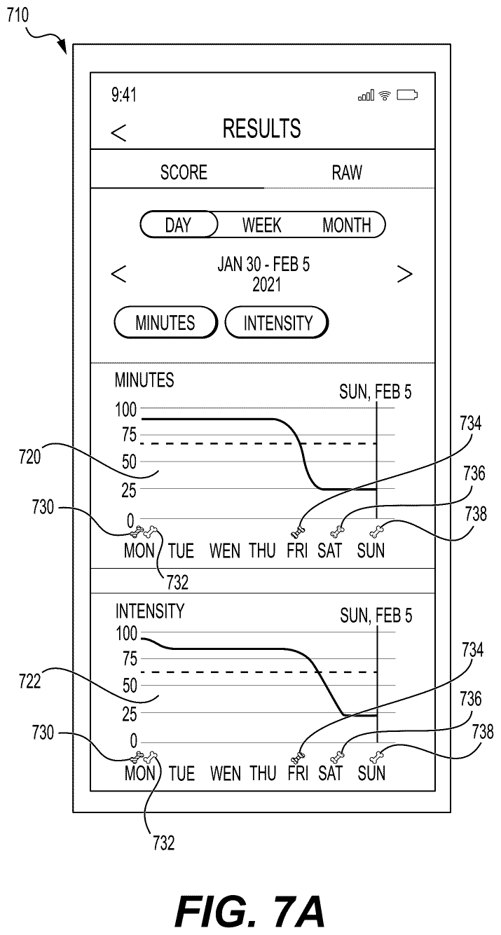

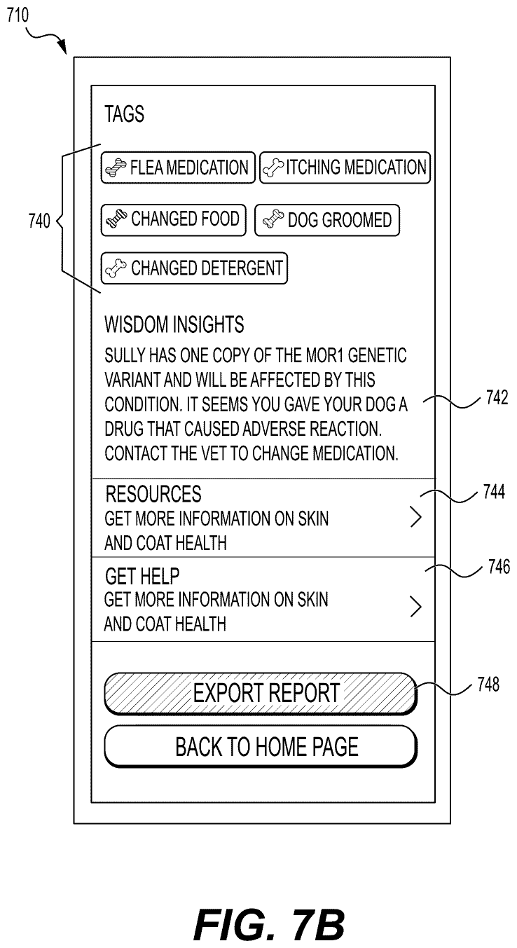

The activity data 244 may be include both raw sensor data received from the smart collar 130 (and/or other components of the wellness system 124) and processed data from one or more models, such as machine learning models. For example, one or more machine learning models may convert the raw sensor data (e.g., accelerometer outputs) from the smart collar 130 into various pet behaviors, such as scratching, licking, walking, lying down, sleeping, eating, drinking and the like, based on historical relationships between historical behavior data and historical activity data. Further, the one or more machine learning models may convert the raw sensor data from the smart collar 130 to mobility data, such as identification of an abnormal gait. The activity data 244 may further include intensity data, such as how intensely and/or vigorously the pet is walking. The activity data 244 may be time series data, such that each data point of the activity data 244 includes both a value corresponding to a determined behavior (e.g., scratching) and a corresponding time stamp. Thus, the activity data 244 may be plotted on a time series plot so that the activity data 244 can be viewed and analyzed to detect changes in activity/behavior over time (see, e.g., FIGS. 5 and 7A).

The pet contextual data 262 may include various types of data relating to the life of the pet. In particular, the pet contextual data 262 may include data observed by the pet owner, including data indicative of a lifestyle of the pet. In some embodiments, the pet contextual data 262 may include, for example, a nutrition regimen of the pet, such as the type(s) of food the pet eats, the quantity of food the pet eats, the time(s) of day the pet eats, treats the pet eats, nutritional supplements the pet takes, and the like. The nutrition regimen may further include changes to the type, quantity, and/or timing of food, treats, nutritional supplements, and the like. In some embodiments, the pet contextual data 262 may include a medication regimen of the pet, such as the type(s) of medication the pet takes, the quantity of medication the pet takes, the time(s) of day the pet takes medication, and the like. The medication regimen may further include changes to the type, quantity, and/or timing of medication. Each individual data point of the pet contextual data 262 may include a time stamp indicating the date and/or time at which the data point was taken.

In some embodiments, the pet contextual data 262 may include one or more life events of the pet. Life events may include an adoption or rehoming, an injury, a change in location (e.g., a move to a new house), a change in the pet's environment (e.g., a new bed), a change in household products (e.g., laundry detergent) used by the pet's owner, a stay at a kennel or boarding facility, a veterinary appointment, and the like. Again, each data point of the pet contextual data 262 may include a time stamp indicating a date and/or time at which the life event(s) occurred.

In some embodiments, the pet contextual data 262 may include mobility state of the pet. Mobility state may include, for example, information related to an injury or medical condition that affects the mobility, stamina, posture, gait, or other motor skills. In some embodiments, mobility state may include limitations or restrictions on the pet's activity during recovery from injury, illness, surgery, or the like.

In some embodiments, the pet contextual data 262 may include mood data indicative of the emotional state of the pet. Mood data may include an indication of whether the pet is happy, sad, lethargic, anxious, etc. Mood data may be based on a subjective assessment by the pet owner, or another individual such as a care provider. Mood data may further include one or more tags indicating a reasoning or justification for a pet owner's (or other individual's) assessment of a particular emotional state. For example, the pet owner may attribute the pet's anxiety to the pet owner being away from home for an extended period of time. Thus, a data point of the mood data indicating that the pet is anxious may include a tag indicating that the pet owner was away from home for an extended period of time. Other examples of tags associated with the mood data include, but are not limited to, an injury, a veterinarian appointment, a trip to a dog park, and the like.

The pet contextual data 262 may be received from an individual, such as the pet owner, via the appware 126 and/or the sensor data correlation system 128 of the wellness system 124 of FIG. 1. In particular, the pet owner may input data into the user interface 129 of the wellness system 124, which is stored as the pet contextual data 262 and may be forwarded to the platform 102 for association with the respective pet profile (e.g., one of pet profiles 204, 206, 208). The pet contextual data 262 may be stored as structured data, unstructured data, or a combination thereof depending on the manner in which the pet contextual data 262 is gathered. Further details of the data format of the pet contextual data 262 are described below with reference to particular embodiments and/or implementations of the present disclosure.

The booking history 252 may include data corresponding to one or more bookings of a third party service (e.g., groomer, trainer, and the like). In some embodiments, the booking history 252 may have been received from one or more of the external systems. For example, the booking history 252 may have been received from the third party services system 182.

### First Exemplary Method

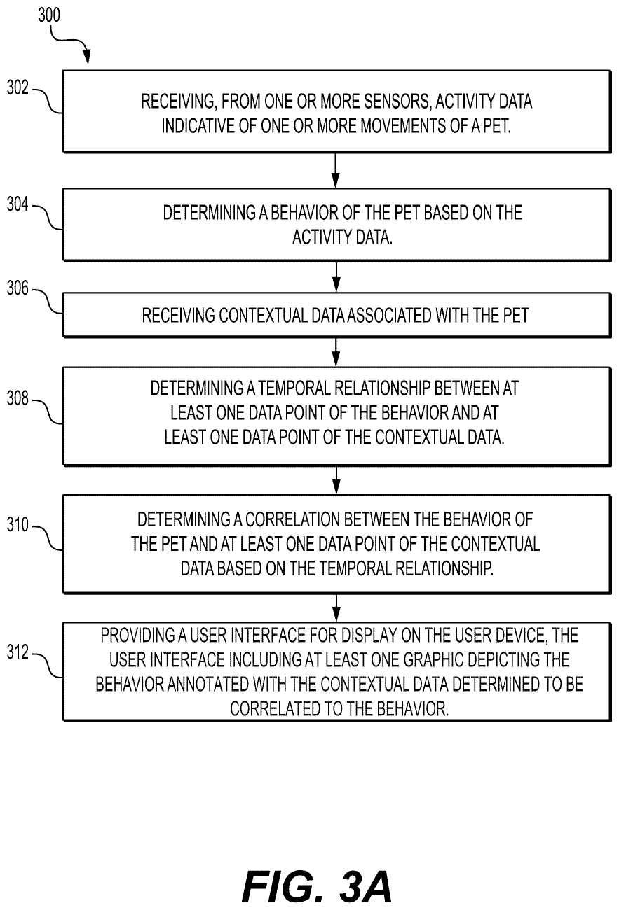

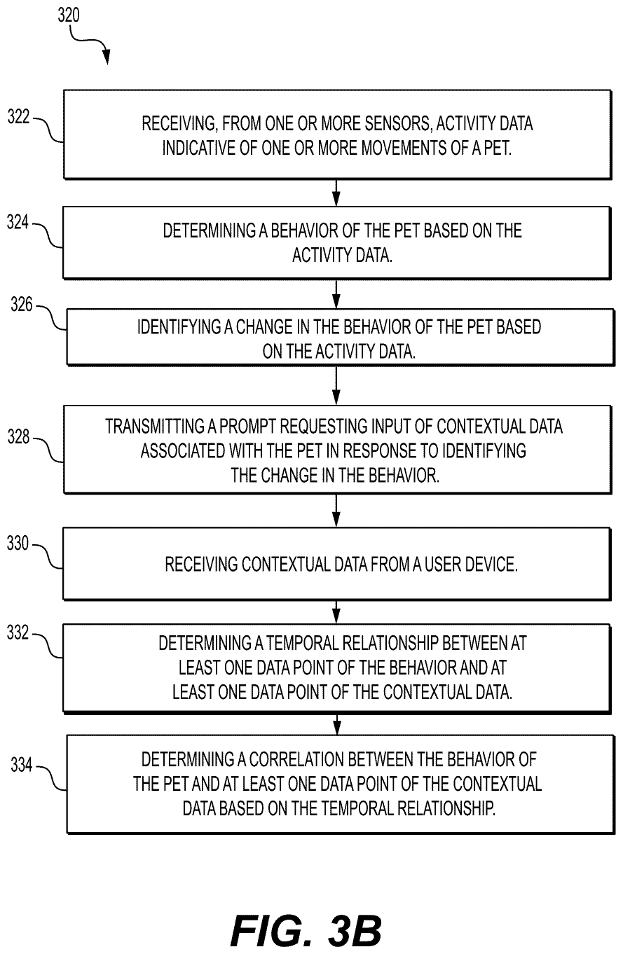

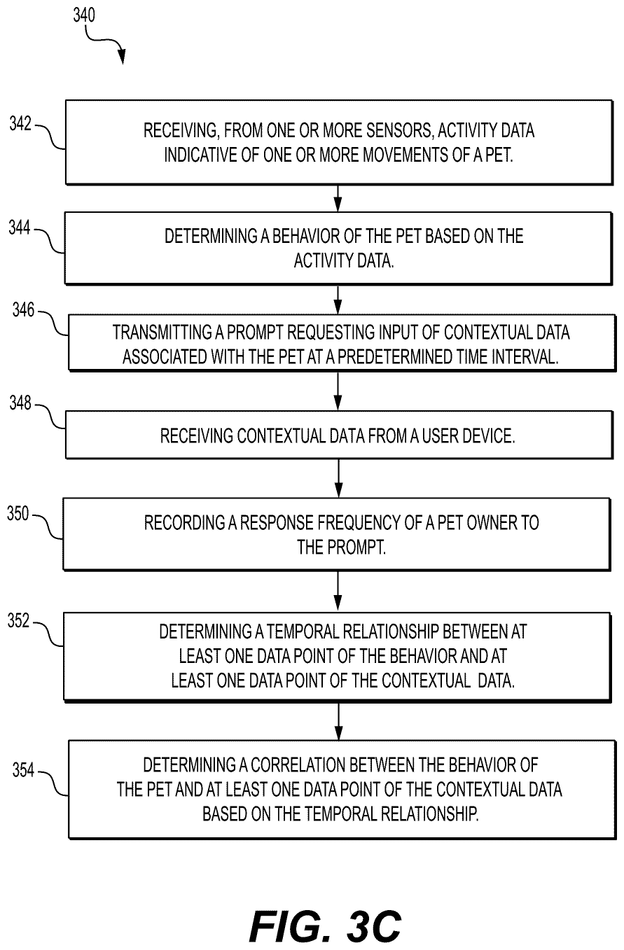

FIG. 3A illustrates an exemplary method 300 for annotating pet health-related sensor data. The method 300 may be performed by one or more processors of a device/server that is in communication with one or more user devices and other external system(s) via a network. That is, each of steps 302-312 of the method 300 may be performed by at least one processor of the environment 100, such as at least one processor associated with the appware 126 and/or the sensor data correlation system 128 of the wellness system 124 and/or at least one processor associated with the platform 102.

The method 300 may include, at step 302, receiving, from one or more sensors, activity data indicative of one or more movements of a pet. The pet may be associated with one of the pet profiles 204, 206, or 208 (see FIG. 2). For simplicity in explaining the method 300, the following description of steps 302-312 will assume that the pet is associated with the pet profile 204. The activity data may include, for example, the activity data 244 (see FIG. 2). The one or more sensors may include, for example, one or more sensors of the smart collar 130 and/or other components of the wellness system 124 (see FIG. 1) configured to generate and/or transmit pet health-related sensor data. The activity data may include a plurality of individual data points, each individual data point including a value indicative of pet behavior and a time stamp.

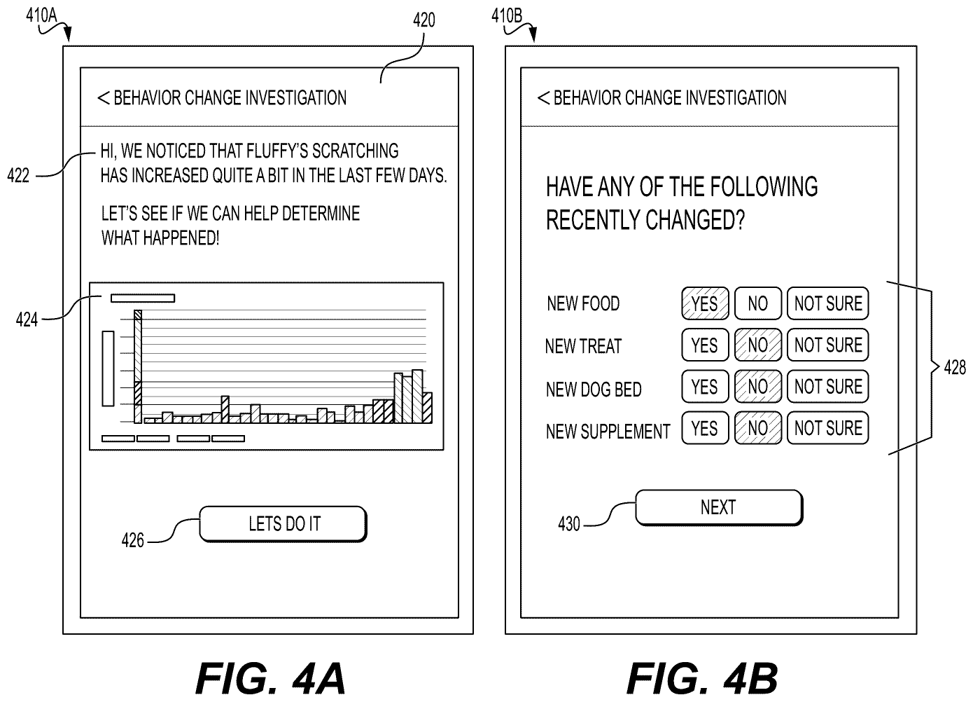

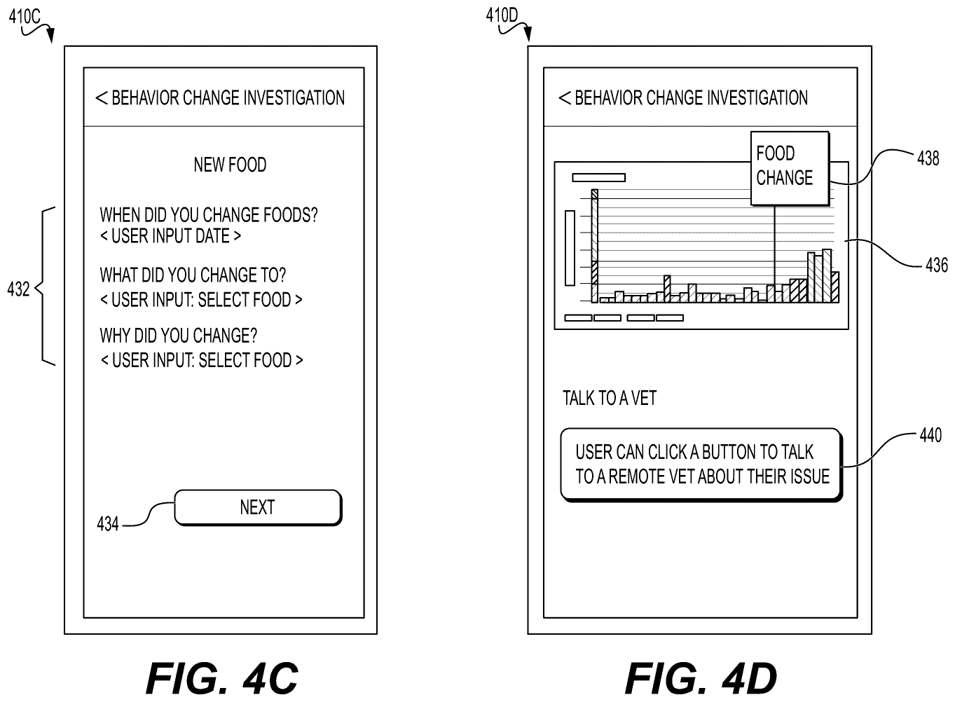

With continued reference to FIG. 3A, the method 300 may further include, at step 304, determining a behavior of the pet based on the activity data. Determining the behavior of the pet may be performed, for example, by applying one or more models (e.g., one or more machine learning models) to raw data received from the smart collar 130. In some embodiments, the behavior may be scratching, licking, walking, lying down, sleeping, eating, drinking, and/or combinations thereof. The behavior of the pet determined at step 304 may be represented as a plurality of data points that may be plotted so that trends in behavior can be observed over time. For example, the behavior may be plotted in a time-series manner (see, e.g., the graph 424 of FIG. 4A and/or the plot 520 of FIG. 5).

With continued reference to FIG. 3A, the method 300 may further include, at step 306, receiving pet contextual data associated with the pet from a user device (e.g., via the user interface 129 displayed on the user device by the appware 126 and/or the user interface 112 associated with the platform 102 of FIG. 1). In some embodiments, the pet contextual data includes a nutrition regimen of the pet, a medication regimen of the pet, a mobility state of the pet, a life event of the pet, and/or a mood (i.e. an emotional state) of the pet. The user device may be any device, e.g. a smart phone, tablet, laptop, desktop and/or wearable device, on which the user interface 129 of FIG. 1 is displayed, such as a smart phone of the owner of the pet associated with the pet profile 204. The pet contextual data may include, for example, the pet contextual data 262 (see FIG. 2). In some embodiments, the pet contextual data may be received indirectly from the user device. For example, the pet contextual data may be received from database(s) 104 (see FIG. 1), which had previously received the pet contextual data from the user device and stored as part of the pet contextual data 262 in the pet profile 204.

The pet contextual data may include a plurality of individual data points, each individual data point including a time stamp. As such, the pet contextual data may be plotted on a time-series plot (see, e.g., plot 520 of FIG. 5) overlaying the behavior data.

In some embodiments, the pet contextual data may be received from the user device substantially contemporaneously with the owner of the user device inputting the pet contextual data into the user device. For example, the pet contextual data may be received upon the owner of the user device responding to a prompt to input pet contextual data into the user device. In some embodiments, the pet contextual data may be received from the user device on a delay. For example, the pet contextual data may be received at predetermined time intervals (e.g., at predetermined times throughout each day). In some embodiments, the pet contextual data may be received from the user device in response to identifying a change in behavior of the pet based on the activity data.

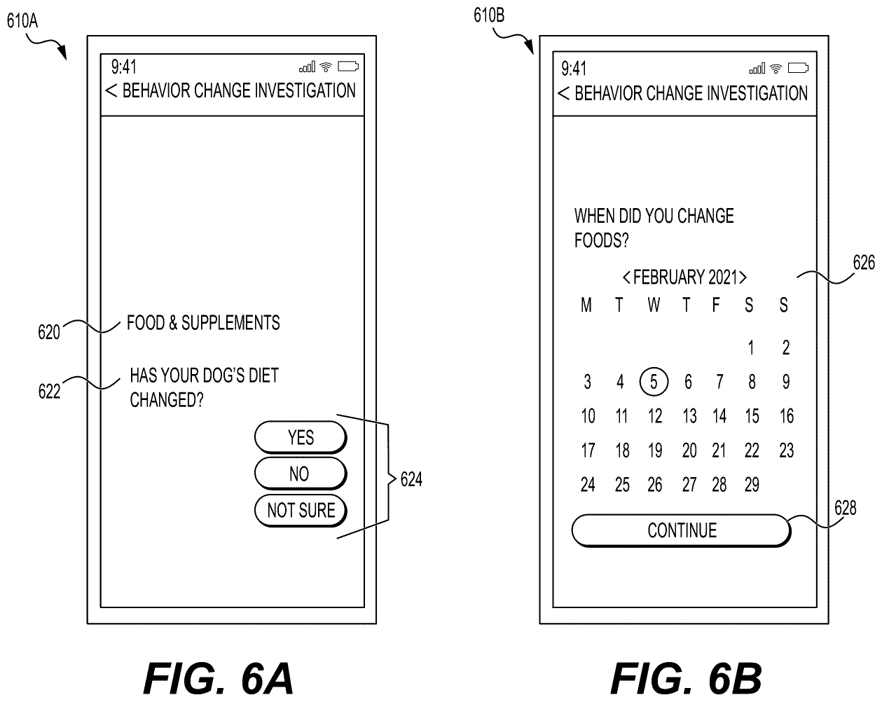

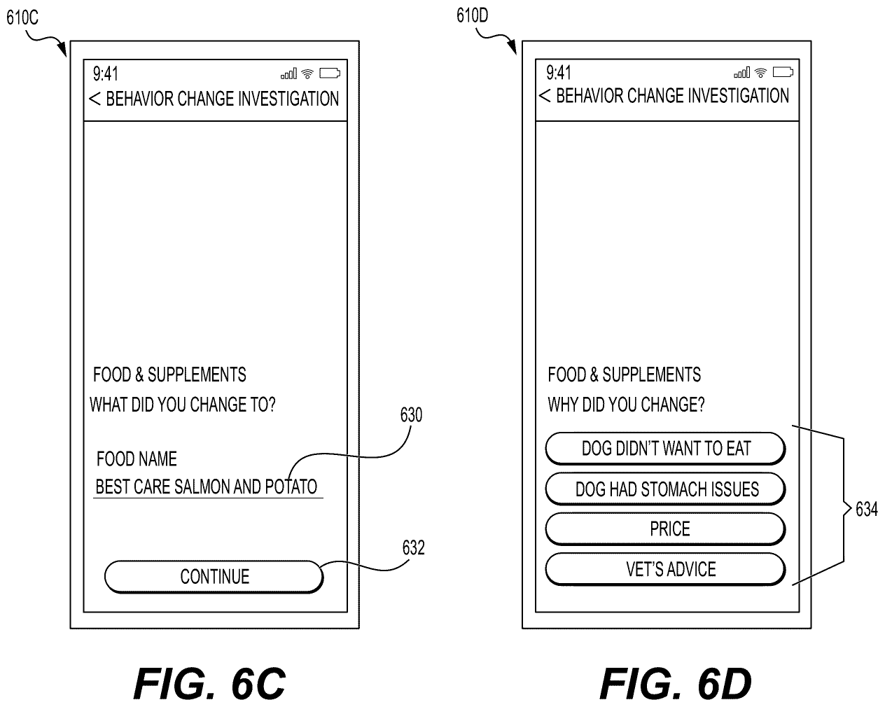

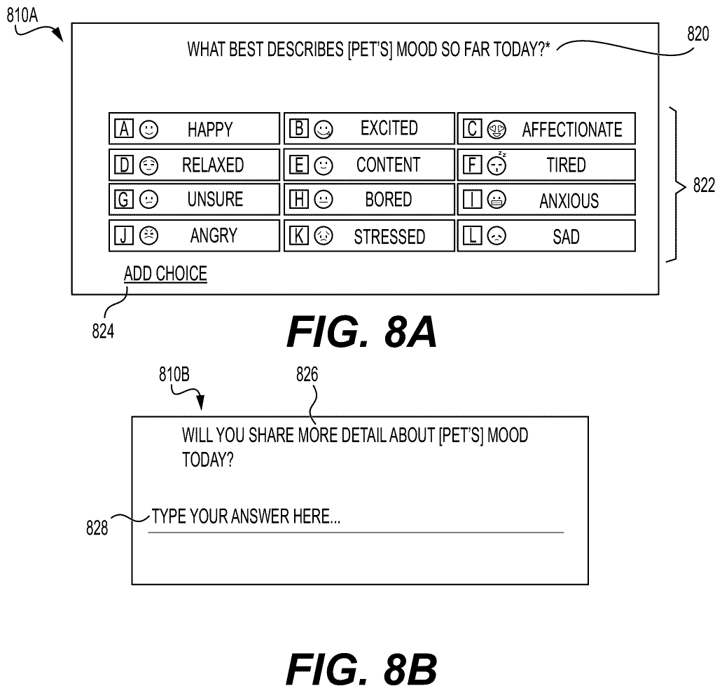

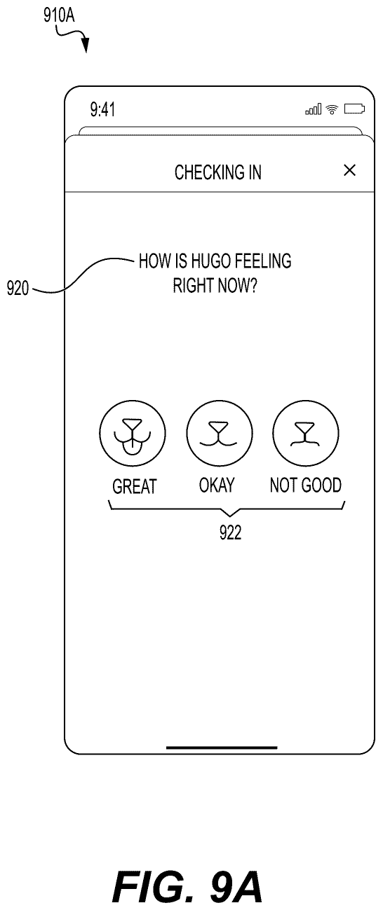

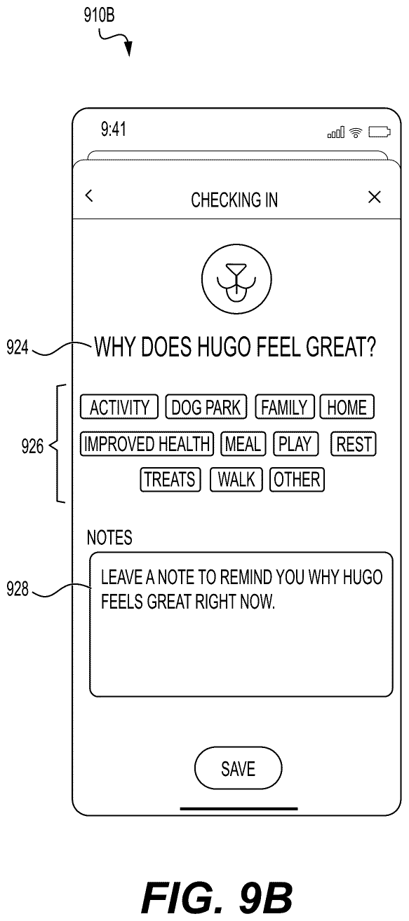

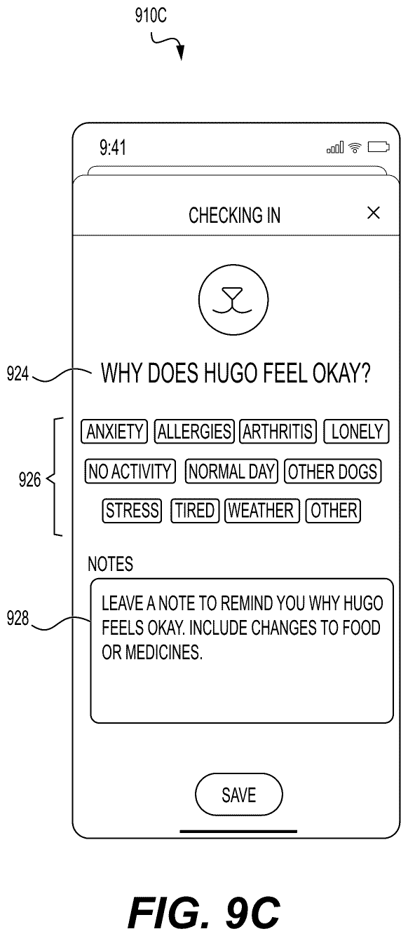

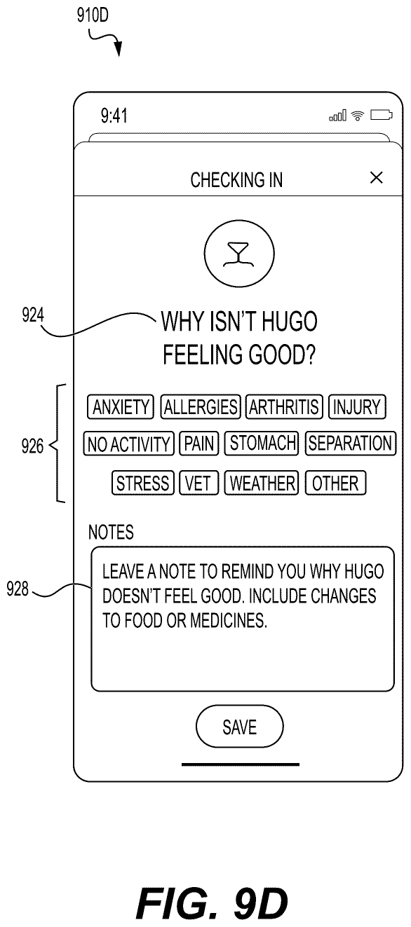

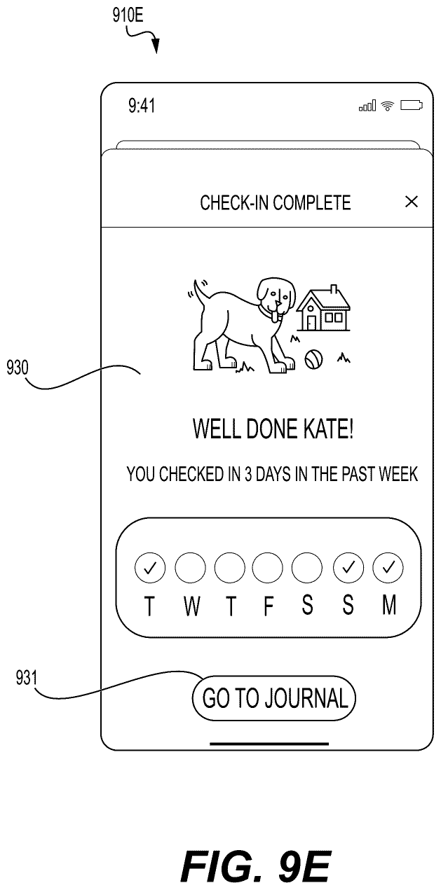

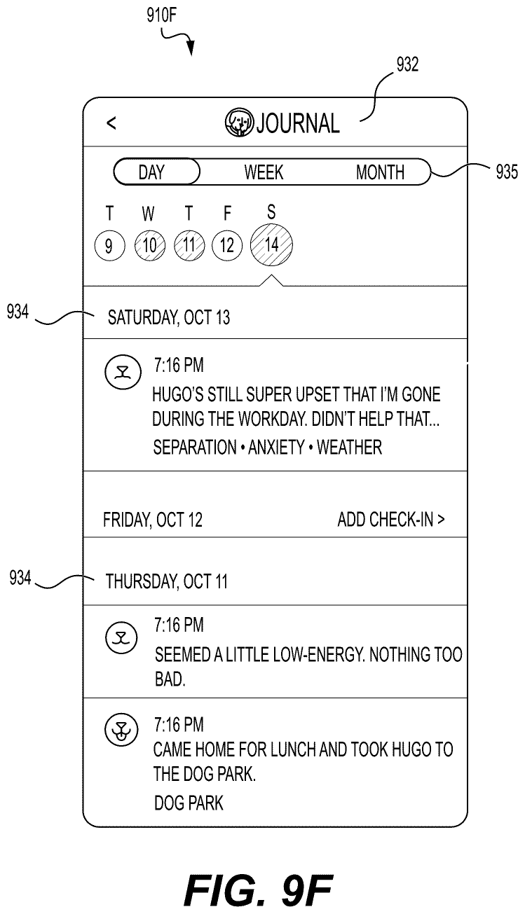

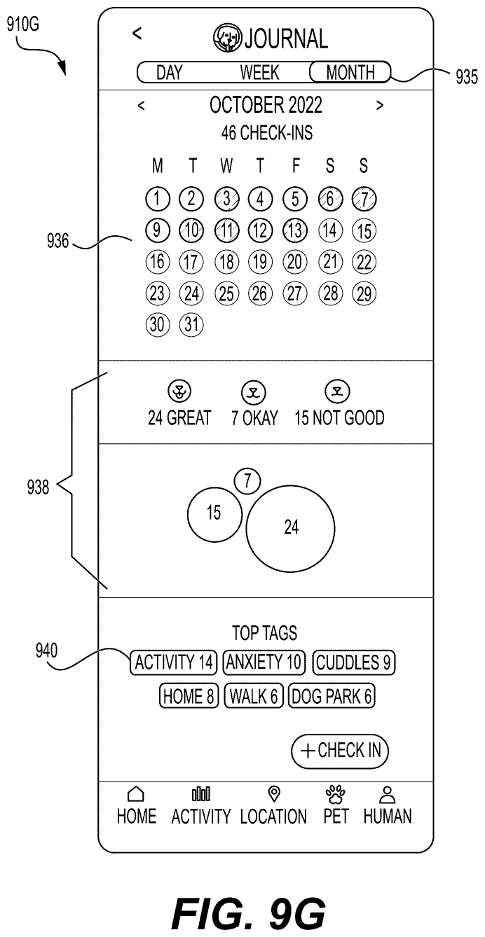

The pet contextual data may include structured data (such as tag selections) and/or unstructured data (such as text input). For example, the pet contextual data may include structured data generated from: pet owner input via option buttons 428 of the user interface 410B of FIG. 4B; pet owner input via the option buttons 624 of the user interface 610A of FIG. 6A; pet owner input via the calendar 626 of the user interface 610B of FIG. 6B; pet owner input via the option buttons 634 of the user interface 61D0 of FIG. 6D; pet owner input via the selectable tags 822 of the user interface 810A of FIG. 8A; and/or pet owner input via the selectable tags 926 of the user interfaces 910B-910D of FIGS. 9B-9D. Similarly, the pet contextual data may include unstructured data generated from: pet owner input via the text field 432 of the user interface 410C of FIG. 4C; pet owner input via the text field 630 of the user interface 610C of FIG. 6C; pet owner input via the text field 828 of the user interface 810B of FIG. 8B; and/or pet owner input via the text field 928 of the user interfaces 910B-910D of FIGS. 9B-9D. In some embodiments, step 306 may include processing unstructured data, for example using one or more machine learning models, into structured data.

With continued reference to FIG. 3A, the method 300 may further include, at step 308, determining a temporal relationship between at least one data point of the behavior and at least one data point of the pet contextual data. The temporal relationship may be based on a relative time difference between a time stamp of a data point of the pet behavior and a time stamp of a data point of the pet contextual data. For example, a temporal relationship may exist between a data point of the pet contextual data and one or more data points of the behavior that have approximately the same time stamp (such as within an hour, within a day, etc.). Further, a temporal relationship may exist between a data point of the pet contextual data and one or more data points of the behavior that occur at substantially different times (such as a difference of multiple days, a week, etc.).

The relationship between data points of the behavior and data points of the pet contextual data may not be one-to-one. For example, the activity data, from which the behavior is determined at step 304, may include a significantly greater number of individual data points compared to the pet contextual data, because the smart collar 130 generates new activity data relatively frequently. For example, the smart collar 130 collects data continuously at sub-second intervals when being worn be the pet, and the collected data is transmitted (e.g. uploaded or synced) to a server or other device in batches for processing. In contrast, new pet contextual data may only be generated when the owner inputs pet contextual data into the user device, which may occur relatively less frequently (such as only daily, or when a significant event in the life of the pet occurs). Further, pet contextual data is reliant on manual data entry by the owner of the pet, and is therefore susceptible to inconsistent and/or omitted entries.

With continued reference to FIG. 3A, the method 300 may further include, at step 310, determining a correlation between the behavior of the pet and at least one data point of the pet contextual data based on the temporal relationship. In some embodiments, the correlation may include that the behavior of the pet and an event associated with the data point of the pet contextual data occurred within a predetermined time of one another, such as within one or more hours, within one or more days, within one or more weeks, etc. The predetermined time may be selected based on the event associated with the data point of pet contextual data. For example, if the event includes administration of a medication, the predetermined time may correspond to a time window in which the medication is expected to take effect. Thus, the correlation is based on data points of the activity data having a temporal relationship to the data point of the pet contextual data that falls within the predetermined time.

Similarly, if the event includes a change in the pet's diet (e.g., a change in type or quantity of food, addition of a nutritional supplement, etc.), the predetermined time may correspond to a time window in which the dietary change may have an observable effect on the pet's behavior. In some embodiments, the predetermined time window may correspond to a time window during which the pet might exhibit symptoms of an allergic reaction caused by the dietary change. In some embodiments, the predetermined time may correspond to a time window during which symptoms of an allergy to a previous food source would be expected to resolve after changing the food.

In some embodiments, determining the correlation at step 310 may include predicting a potential cause of a change in the behavior of the pet based on the pet contextual data. For example, the prediction may be performed using one or more machine learning models trained on historical relationships between historical pet contextual data, historical activity data, and historical diagnoses. In some embodiments, the method 300 may include, prior to step 310, identifying a change in the behavior of the pet based on the activity data. The change in the behavior may be identified based on observed trends in the activity data. For example, the activity data may indicate that the pet exhibited an increase or decrease in a behavior, such as scratching, licking, walking, lying down, eating, drinking, etc. The prediction of the potential cause of the change in the behavior may be based on the temporal relationship between one or more data points of the pet contextual data and a time of the behavior change. For example, the data point of the pet contextual data may indicate a dietary change, and may have a temporal relationship with the pet behavior indicating an increase in scratching. Thus, the prediction may include that the dietary change is a potential cause of the increased scratching, based on the increased scratching beginning within a time window for which dietary-induced scratching would be expected to occur. As another example, the data point of the pet contextual data may indicate administration of medication, and may have a temporal relationship with the pet behavior indicating a decrease in scratching. Thus, the prediction may include that the medication is a potential cause of the decreased scratching, based on the decreased scratching beginning within an effective time window of the medication.

With continued reference to FIG. 3A, the method 300 may further include, at step 312, providing a user interface for display on the user device (such as the user interface 129 of the wellness system 124 and/or the user interface 112 associated with the platform 102 of FIG. 1). The user interface includes at least one graphic depicting the behavior annotated with the contextual data determined to be correlated with the behavior. The at least one graphic may include various elements of the user interfaces 410A-910G described herein. In some embodiments, the graphic may include a plot, chart, or graph, such as a plot 520 as in the user interface 510 of FIG. 5, or a graph 720, 722 as in the user interface 710 of FIG. 7A.

Although FIG. 3A shows example blocks of exemplary method 300, in some implementations, the exemplary method 300 may include additional blocks, fewer blocks, different blocks, or differently arranged blocks than those depicted in FIG. 3A. Additionally, or alternatively, two or more of the blocks of the exemplary method 300 may be performed in parallel, where context would allow.

### Second Exemplary Method

FIG. 3B illustrates an exemplary method 320 for annotating pet health-related sensor data. The method 320 may be performed by one or more processors of a device/server that is in communication with one or more user devices and other external system(s) via a network. That is, each of steps 322-334 of the method 320 may be performed by at least one processor of the environment 100, such as at least one processor associated with the appware 126 and/or the sensor data correlation system 128 of the wellness system 124 and/or at least one processor associated with the platform 102.

The method 320 may include, at step 322, receiving, from one or more sensors, activity data indicative of one or more movements of a pet. The pet may be associated with one of the pet profiles 204, 206, or 208 (see FIG. 2). The method 320 may include, at step 324, determining a behavior of the pet based on the activity data. Steps 322 and 324 of the method 320 may be substantially identical to steps 302 and 304 of the method 300 of FIG. 3A.

With continued reference to FIG. 3B, the method 320 may further include, at step 326, identifying a change in the behavior of the pet based on the activity data. The change in the behavior may be identified based on observed trends in the activity data. For example, the activity data may indicate that the pet exhibited an increase or decrease in a behavior, such as scratching, licking, walking, laying down, etc.

With continued reference to FIG. 3B, the method 320 may further include, at step 328, transmitting a prompt requesting input of contextual data associated with the pet in response to identifying the change in the behavior. The prompt may be displayed on a user interface (e.g., the user interface 410B of FIG. 4B) of the user device. The prompt may include a list of selectable options, where each of the plurality of selectable options corresponds to a life event of the pet. In some examples, the life event may be a particular type of event known to often cause behavioral changes of the type identified. The selectable options may include, for example, the options buttons 428 of the user interface 410B of FIG. 4B.

With continued reference to FIG. 3B, the method 320 may further include, at step 330, receiving the contextual data associated with the pet from a user device (e.g., via the user interface 129 displayed on the user device by the appware 126 and/or the user interface 112 associated with the platform 102 of FIG. 1). Step 330 may be substantially identical to step 306 of method 300 of FIG. 3A. In particular, the pet contextual data may be the data input by the pet owner in response to transmission of the prompt at step 328. That is, the pet contextual data may include the pet owner's selection of one or more of the option buttons 428 of the user interface 410B of FIG. 4B.

With continued reference to FIG. 3B, the method 320 may further include, at step 332, determining a temporal relationship between at least one data point of the behavior and at least one data point of the contextual data. Step 332 may be substantially identical to step 308 of the method 300 of FIG. 3A.

With continued reference to FIG. 3B, the method 320 may further include, at step 334, determining a correlation between the behavior of the pet and at least one data point of the contextual data based on the temporal relationship. Step 334 may be substantially identical to step 310 of the method 300 of FIG. 3A. In some embodiments, the correlation may specifically be determined between the change in the behavior of the pet identified at step 326 and at least one data point of the contextual data.

Although FIG. 3B shows example blocks of exemplary method 320, in some implementations, the exemplary method 320 may include additional blocks, fewer blocks, different blocks, or differently arranged blocks than those depicted in FIG. 3B. Additionally, or alternatively, two or more of the blocks of the exemplary method 320 may be performed in parallel, where context would allow.

### Third Exemplary Method

FIG. 3C illustrates an exemplary method 340 for annotating pet health-related sensor data. The method 340 may be performed by one or more processors of a device/server that is in communication with one or more user devices and other external system(s) via a network. That is, each of steps 342-354 of the method 340 may be performed by at least one processor of the environment 100, such as at least one processor associated with the appware 126 and/or the sensor data correlation system 128 of the wellness system 124 and/or at least one processor associated with the platform 102.

The method 340 may include, at step 342, receiving, from one or more sensors, activity data indicative of one or more movements of a pet. The pet may be associated with one of the pet profiles 204, 206, or 208 (see FIG. 2). The method 340 may include, at step 344, determining a behavior of the pet based on the activity data. Steps 342 and 344 of the method 340 may be substantially identical to steps 302 and 304 of the method 300 of FIG. 3A.

With continued reference to FIG. 3C, the method 340 may further include, at step 346, transmitting a prompt requesting input of contextual data associated with the pet at a predetermined time interval. The time interval may be, for example, daily, twice daily, every other day, weekly, etc. The prompt may be displayed on a user interface (e.g., the user interface 910A of FIG. 9A) of the user device. The prompt may include, for example, a message 920 of the user interface 910A of FIG. 9A.

With continued reference to FIG. 3C, the method 340 may further include, at step 348, receiving the contextual data associated with the pet from a user device (e.g., via the user interface 129 displayed on the user device by the appware 126 and/or the user interface 112 associated with the platform 102 of FIG. 1). Step 348 may be substantially identical to step 306 of method 300 of FIG. 3A. In particular, the pet contextual data may be the data input by the pet owner in response to transmission of the prompt at step 346. That is, the pet contextual data may include the pet owner's selection of one the option buttons 922 of the user interface 910A of FIG. 9A; the pet owner's selection of one or more of the selectable tags 926 of the user interfaces 910B-910D of FIGS. 9B-9D; and/or the pet owner's input via the text field 928 of the user interfaces 910B-910D of FIGS. 9B-9D.

With continued reference to FIG. 3C, the method 340 may further include, at step 350, recording a response frequency of a pet owner to the prompt transmitted at step 346. The response frequency may be representative of, for example, the rate at which the pet owner responds to the prompt transmitted at step 346. That is, each time the prompt is transmitted at step 346, in accordance with the predetermined time interval, the response frequency may be updated based on whether or not the pet owner input pet contextual data (e.g., the emotional state of the pet) in response to the prompt. The response frequency may be utilized to generate and display a graphic on the user interface of the user device. For example, the response frequency may be used to generate and display the message 930, the digital journal 932, the calendar 936, the graphic 938, and/or the graphic 940 of the user interfaces 910E-910G of FIGS. 9E-9G.

With continued reference to FIG. 3C, the method 340 may further include, at step 352, determining a temporal relationship between at least one data point of the pet behavior and at least one data point of the contextual data. Step 352 may be substantially identical to step 308 of the method 300 of FIG. 3A.

With continued reference to FIG. 3C, the method 340 may further include, at step 354, determining a correlation between the behavior of the pet and at least one data point of the contextual data based on the temporal relationship. Step 354 may be substantially identical to step 310 of the method 300 of FIG. 3A.

In some embodiments, the method 340 may further include providing a user interface for display on the user device (such as the user interface 129 of the wellness system 124 and/or the user interface 112 associated with the platform 102 of FIG. 1). In some embodiments, the user interface may present a digital journal including mood data received from the user device, as part of the contextual data, at step 348. For example, the digital journal may include a plurality of entries including the mood data of the pet, each entry including a time stamp corresponding to a day and/or time that the mood data was received. The digital journal may include, for example, the digital journal 932 of the user interface 910F of FIG. 9F. In some embodiments, the user interface may include a calendar indicating one or more days on which mood data was received from the user device. The calendar may include, for example, the calendar 936 of the user interface 910G of FIG. 9G. In some embodiments, the user interface may include a chart indicating a frequency at which particular types of mood data were received from the user device. For example, the chart may indicate how frequently mood data types such as “Great”, “Ok”, and “Not Good” were received from the user device. The chart may include, for example, the graphic 938 of the user interface 910G of FIG. 9G.

Although FIG. 3C shows example blocks of exemplary method 340, in some implementations, the exemplary method 340 may include additional blocks, fewer blocks, different blocks, or differently arranged blocks than those depicted in FIG. 3C. Additionally, or alternatively, two or more of the blocks of the exemplary method 340 may be performed in parallel, where context would allow.

### Exemplary Implementations

FIGS. 4A-4D depict a plurality of exemplary user interfaces 410A, 410B, 410C, 410D that may be utilized with the techniques presented herein. Each of the plurality of user interfaces 410A, 410B, 410C, 410D may be displayed on a user device (e.g., a user device associated with the user interface 129 of the wellness system 124 and/or the user interface 112 associated with the platform 102 of FIG. 1) to allow a user (e.g., a pet owner) to interact with the wellness system 124 and/or the platform 102. The user interfaces 410A-410D may display various prompts, messages, or the like to the user according to the various techniques described herein.

As illustrated in FIG. 4A, the user interface 410A may display a notification 420. The notification 420 may be generated and displayed by at least one processor in response to various systems of environment 100 (see FIG. 1) receiving and/or generating data. For example, the notification 420 shown in FIG. 4A may be generated in response to the wellness system 124 (see FIG. 1) detecting that the pet (e.g., a pet associated with the profile 204) is exhibiting a behavior change, such as unusual and/or excessive scratching, corresponding to step 326 of the method 320 of FIG. 3B. Particularly, the smart collar 130 and/or smart camera 138 may detect the unusual and/or excessive scratching, and the wellness system 124 may automatically provide updated activity data 244 to the platform 102 for storage in association with the pet profile 204 to reflect this behavior. In response to the activity data 244 being updated, the appware 126, the sensor data correlation system 128, and/or the platform 102 may generate and cause display of the notification 420 on the user interface 410A, corresponding to step 328 of the method 320 of FIG. 3B. In some examples, the appware 126, the sensor data correlation system 128, and/or the platform 102 may cause display of the notification 420 on the user interface 410A upon a user's next access of an application associated with the appware 126 running on the user device and/or the user's next access of the platform 102.

The notification 420 may include a message 422 describing the behavior change detected by the wellness system 124 and included in activity data 244 (see FIG. 2). For example, in FIG. 4A, the message states “Hi, we noticed that Fluffy's scratching has increased quite a bit in the last few days. Let's see if we can help determine what happened”. The notification 420 may further include a visual representation of the behavior change, such as a graph 424 indicating the daily frequency of a behavior (e.g., scratching) derived and/or generated from activity data 244.

The notification 420 may further include a control element, such as a button 426, allowing the user to proceed with investigation of the behavior change. By selecting the button 426, the user interface 410B may display a plurality of option buttons 428 corresponding to various pet lifestyle changes, such as a new food, new treat, new bed, and new supplement, as shown in FIG. 4B. A subset of the option buttons 428 may be associated with each pet lifestyle change. The user may select an option (e.g., “Yes”, “No”, or “Not Sure”) from the subset of the option buttons 428 associated with each lifestyle change. In the illustrated example, the user has indicated that the pet's food has changed—but that the pet's treats, bed, and supplements have not—by selecting the corresponding option buttons 428. The information entered by the user (i.e., the selection of the option buttons 428) may be stored as the pet contextual data 262 (see FIG. 2). In particular, this information may be stored as structured data because the information indicated by the selection of the option buttons 428 is discrete and does not require interpretation/transformation. The user interface 410B may further include a control element, such as button 430, allowing the user to proceed.

Upon selecting the button 430, the user interface 410C may display one or more text fields 432 prompting the user to input details of the pet lifestyle change indicated by the user, as shown in FIG. 4C. For example, when the user has indicated that the pet's food has changed, the one or more text fields 432 may prompt the user to enter a time and/or day when the pet food was changed, a type of the new pet food, and a reason for the change of pet food. The information entered by the user (i.e., information input into the text fields 432) may be stored as the pet contextual data 262 (see FIG. 2). In particular, information in the text field may be stored as unstructured data that may be subsequently processed to extract pertinent information. For example, one or more natural language processing (NLP) machine learning models may be utilized to convert the unstructured data into a format suitable for further analysis and/or comparison (e.g., to structured data). In some embodiments, the unstructured data may be formatted according to a predefined classification or taxonomy system. The user interface 410C may further include a button 434 allowing the user to proceed.

Upon selecting the button 434, the user interface 410D may display a visual representation of the behavior change correlated to the pet lifestyle change, as shown in FIG. 4D. The visual illustration may include a graph 436 indicating the daily frequency of the behavior for which the change was detected (e.g., scratching) that is overlaid with the pet lifestyle information received from the user. For example, the graph 436 may include a notation 438 indicating the date of the food change, so that the user can observe any correlation between the timing of the food change and the increased incidence of scratching detected by the wellness system 124. The user interface 410D may further include a chat feature 440 allowing the user to interact with a health professional, such as a veterinarian, via the diagnostic system 142 (see FIG. 1).

FIG. 5 depicts another exemplary user interface 510 that may be generated and/or utilized with the techniques presented herein. The user interface 510 may be displayed on a user device (e.g., a user device associated with the user interface 129 of the wellness system 124 or the user interface 112 associated with the platform 102 of FIG. 1) to allow a user (e.g., a pet owner) to interact with the wellness system 124 via the appware 126 and/or the platform 102. The user interface 510 may display various prompts, messages, or the like to the user according to the various techniques described herein.

User interface 510 may display a visual representation of a pet's behavior over time notated with life events. For example, the visual representation may include a time series data plot 520 of one or more behaviors, such as scratching, shown by curve 522, and licking, shown by curve 524. The time series data plot 520 may be derived and/or generated from activity data 244 (see FIG. 2). The time series data plot 520 may further include annotations of pet life events, among other types of contextual data, contained in pet contextual data 262 (see FIG. 2). For example, the annotations of pet life events may include a diet change annotation 526 and a medication change annotation 528. The diet change annotation 526 indicates a day on which a change to the pet's diet (e.g., type and/or quantity of food, treats, supplements, etc.) was made. Similarly, the medication change annotation 528 indicates a day on which a change to the pet's medication was made. By overlaying annotations 526, 528 onto time series data plot 520, the user may appreciate the temporal relationship and/or correlation between the pet lifestyle changes and pet activity data 244.

FIGS. 6A-6D depict another plurality of exemplary user interfaces 610A, 610B, 610C, 610D that may be generated and/or utilized with the techniques presented herein. The user interfaces 610A-610D may be displayed on a user device (e.g., a user device associated with the user interface 129 of the wellness system 124 and/or the user interface 112 associated with the platform 102 of FIG. 1) to allow a user (e.g., a pet owner) to interact with the wellness system 124 via the appware 126 and/or the platform 102. The user interface may display various prompts, messages, or the like to the user according to the various techniques described herein.

As illustrated in FIG. 6A, the user interface 610A may display a notification 620. The notification 620 may be generated and displayed by at least one processor in response to various systems of environment 100 of FIG. 1 receiving and/or generating data. For example, the notification 620 shown in FIG. 6A may be generated in response to the wellness system 124 (see FIG. 1) determining that the pet (e.g., a pet associated with the profile 204) is exhibiting a behavior change, such as unusual and/or excessive scratching, corresponding to step 326 of the method 320 of FIG. 3B. Particularly, the smart collar 130 and/or smart camera 138 may detect the unusual and/or excessive scratching, and the wellness system 124 may automatically provide updated activity data 244 to the platform 102 for storage in association with the pet profile 204 to reflect this behavior. In response to the activity data 244 being updated, the appware 126, the sensor data correlation system 128, and/or the platform 102 may generate and cause a display of the notification 620 on the user interface 610A, corresponding to step 328 of the method 320 of FIG. 3B. In some examples, the notification 620 may be displayed on the user interface 610A upon a user's next access of an application associated with the appware 126 running on the user device and/or the user's next access of the platform 102.

The notification 620 may include a message 622 asking whether the pet has experienced a lifestyle change, such as a change in food/diet, as shown in FIG. 6A. The notification 620 may include one or more option buttons 624 facilitating a response to the message 622, such as “Yes”, “No”, and “Not Sure”. If the user selects the option button 624 associated with “Yes”, the user interface 610B may display a calendar 626 or other input graphic allowing the user to input the date of the food change, as shown in FIG. 6B. The date may be stored as pet contextual data 262, particularly as structured data. The user interface 610B may further include a button 628 allowing the user to proceed after inputting the date into the calendar 626.

Upon selecting the button 628, the user interface 610C may display a text field 630 prompting the user to input details of the pet lifestyle change indicated by the user, as shown in FIG. 6C. For example, when the user indicates a change of pet food, the text field 630 may prompt the user to enter a type of the new pet food, and a reason for the change of pet food. In some embodiments, the text field 630 may allow the user to select from a list of preconfigured inputs (e.g., a list of commercially available pet foods. In some embodiments, the text field 630 may allow the user to enter unstructured data, i.e. free form text. The data input into the text field 630 may be stored as pet contextual data 262. The user interface 610C may further include a button 632 allowing the user to proceed after inputting the date into the calendar 626.

Upon selecting the button 632, the user interface 610D may display a message and a plurality of option buttons 634 prompting the user to input a reason for the lifestyle change, as shown in FIG. 6D. For, example the option buttons may correspond to “Dog did not eat”, “Dog had stomach issues”, “Price”, and “Not sure”. The information input by the user (i.e., the selection of one or more of the option buttons 634) may be stored as pet contextual data 262 (see FIG. 2), particularly structured data.

FIGS. 7A-7B depict another exemplary user interface 710 that may be generated and/or utilized with the techniques presented herein. The user interface 710 may be displayed on a user device (e.g., a user device associated with the user interface 129 of the wellness system 124 and/or the user interface 112 associated with the platform 102 of FIG. 1) to allow a user (e.g., a pet owner) to interact with wellness system 124 via the appware 126 and/or the platform 102. The user interface 710 may display various prompts, messages, or the like to the user according to the various techniques described herein.

As shown in FIG. 7A, a first portion of the user interface 710 may include one or more visual representations of pet behavior and/or activity based on the activity data 244 (see FIG. 2). For example, the first portion of the user interface 710 of FIG. 7A includes a graph 720 of time spent (in minutes) performing a behavior/activity (e.g., scratching) each day of a week (e.g., Monday, January 30 through Sunday, February 5 as illustrated), and a graph 722 of intensity of that activity each day of the same week. Each of the graphs 720, 722 may include annotations 730, 732, 734, 736, 738 indicating pet life events, such as starting a medication regimen, changing food, etc. that occurred on various days of the week included in the graphs 720, 722. A second portion of the user interface 710, shown in FIG. 7B, may further include a legend 740 providing a description to the annotations 730-738. For example, FIGS. 7A-7B show a drop-off in time and intensity of the behavior shown in the graphs 720, 722, respectively, which correspond to a food change that occurred on Friday, February 3. Additionally, beginning a medication regimen of flea medication and itching medication on Monday, January 30 does not correspond to an immediate drop in time spend performing the behavior, but does correspond to a slight drop in intensity of the behavior.

As shown in FIG. 7B, the second portion of the user interface 710 may further include a message 742 including genetic information and/or analysis corresponding to the activity data 244 and/or the pet contextual data 262 presented in the graphs 720, 722. The genetic information and/or analysis may be generated by genetics system 170 (see FIG. 1). The second portion of the user interface 710 may further include a button 744 for obtaining resources, such as articles about the pet's behaviors from the content management system 164 (see FIG. 1). The user interface 710 may further include a button 746 for obtaining help, such as by contacting a veterinarian or behaviorist via the diagnostic system 142 (see FIG. 1). For example, selection of the button 746 may initiate an expert interaction 147. The user interface 710 may further include a button 748 for exporting a report including the activity data 244 and/or the pet contextual data 262 presented in the graphs 720, 722 in a file (e.g. a .xlsx file, a .xls fil, a .csv file, or the like).

FIGS. 8A-8B depict another plurality of exemplary user interfaces 810A, 810B that may be generated and/or utilized with the techniques presented herein. The user interfaces 810A, 810B may be displayed on a user device (e.g., a user device associated with the user interface 129 of the wellness system 124 and/or the user interface 112 associated with the platform 102 of FIG. 1) to allow a user (e.g., a pet owner) to interact with the wellness system 124 via the appware 126 and/or the platform 102. The user interfaces 810A, 810B may display various prompts, messages, or the like to the user according to the various techniques described herein.

As shown in FIG. 8A, the user interface 810A may include a first message 820 prompting the user to input a mood (i.e. emotional state) of the pet. The user interface 810A may further include a plurality of selectable tags 822 corresponding to various moods such as “Happy”, “Excited”, “Affectionate”, “Relaxed”, etc. The user interface 810A may further include a button 824 allowing the user to add a user-generated mood not included among the selectable tags 822. As shown in FIG. 8B, the user interface 810B may further include a second message 826 prompting the user to input additional details about the mood of the pet in a text field 828. For example, the user may enter into the text field 828 a life event (e.g., a veterinary appointment) that the user believes triggered the pet's mood. The information input by the user via the selectable tags 822 and/or the text field 828 may be stored as pet contextual data 262. In particular, the information received via the selectable tags 822 may be structured data, and the information received via the text field 828 may be stored as unstructured data.

In some embodiments, one or more of the user interfaces 810A, 810B may be automatically displayed on the user device at predetermined time intervals, such as at a predetermined time every day, to prompt the user participation on a continual basis to expand the amount of information contained in pet contextual data 262. Automatic display of one or more of the user interfaces 810A, 810B may correspond to, for example, step 346 of the method 340 of FIG. 3C.

FIGS. 9A-9G depict another plurality of exemplary user interfaces 910A, 910B, 910C, 910D, 910E, 910F, 910G that may be generated and/or utilized with the techniques presented herein. The user interfaces 910A-910G may be displayed on a user device (e.g., a user device associated with the user interface 129 of the wellness system 124 and/or the user interface 112 associated with the platform 102 of FIG. 1) to allow a user (e.g., a pet owner) to interact with the wellness system 124 via the appware 126 and/or the platform 102. The user interfaces 910A-910G may display various prompts, messages, or the like to the user according to the various techniques described herein.

As shown in FIG. 9A, the user interface 910A may include a message 920 prompting the user to input a mood (i.e. an emotional state) of the pet. The user interface 910A may further include a plurality of option buttons 922 corresponding to various emotional states such as “Great”, “Okay”, “Not good”, or the like. Upon selection of one of the option buttons 922, a corresponding one of the user interfaces 910B, 910C, or 910D may display a second message 924 prompting the user to input additional information such as a one or more justifications for the pet's emotional state, as shown in FIGS. 9B-9D.

Each of the user interfaces 910B-910D may include a plurality of selectable tags 926 corresponding to various justifications for the particular emotional state selected from user interface 910A. For example, as shown in FIG. 9B, the tags 926 corresponding to a selected emotional state of “Great” may include “Activity” (i.e., the pet received a significant amount of physical stimulation), “Dog park” (i.e., the pet went to a dog park), “Family” (i.e., the pet spent time with family), etc. As shown in FIG. 9C, the tags 926 corresponding to a selected emotional state of “Okay” may include “Anxiety”, “Allergies”, “Arthritis”, etc. As shown in FIG. 9D, the tags 926 corresponding to a selected emotional state of “Not good” may include “Anxiety”, “No activity”, “Stress”, etc. Referring still to FIGS. 9B-9D, each of the user interfaces 910B-910D may further include a text field 928 where the user may enter additional description and/notes related to the pet's emotional state.

The information input via tags 926 and text field 928 may be stored as pet contextual data 262 (see FIG. 2). In particular, the information received via the tags 926 may be structured data, and the information received via the text field 928 may be stored as unstructured data. The information may be stored in association with a time stamp indicating a date and/or time the information was provided as input.

In some embodiments, one or more of the user interfaces 910A-9D of FIGS. 9A-9D may be automatically displayed on the user device at predetermined time intervals, such as at a predetermined time every day, to prompt user participation on a continual basis to expand the amount of information collected and stored as pet contextual data 262. Automatic display of one or more of the user interfaces 910A-9D may correspond to, for example, step 346 of the method 340 of FIG. 3C.

As shown in FIG. 9E, the user interface 910E may include a message 930 displaying participation statistics of the user, such as a number of days in the past week that the user has provided information relating to the mood or emotional state of the pet. The message 930 may be configured to encourage participation by the user in order to maintain consistent and meaningful data collection for pet contextual data 262. For example, the message 930 may include positive feedback if the user has input data on a predetermined number of days in the current week. The user interface 910E may further include a button 931, selection of which causes the user interface 910F to display a digital journal 932 summarizing the information input by the user each time the user interacted with the user interfaces 910A-910D. As shown in FIG. 9F, the digital journal 932 of the user interface 910F may include, for example, data relating to the emotional state of the pet displayed in association with time stamps 934 corresponding to the time at which the user input the data relating to the emotional state. The user interface 910F may further include a control element, such as a toggle button 935, that allows the user to switch between various displays, such as between the user interface 910F and the user interface 910G. In FIG. 9F, the toggle button 935 is set to “Day”, which displays the digital journal 932 as shown in the user interface 910F.

Referring now to FIG. 9G, the user interface 910G may be configured to display a calendar 936. The calendar 936 may visually indicate which days the user input pet contextual data 262. For example, days on which the user input pet contextual data 262 may be identified by a symbol or different color relative to other days. The user interface 910G may also be configured to display a graphic 938 (e.g., a chart) illustrating the frequency at which the user input each emotional state of the pet (i.e. “Great”, “OK”, and “Not Good”) (see FIG. 9A). The user interface 910G may also be configured to display a graphic 940 illustrating the most frequently used tags 926 (see FIGS. 9B-9D) input by the user. The user interface 910G may further include the toggle button 935 (as included in the user interface 910F of FIG. 9F) to allow the user to switch between various displays. In FIG. 9G, the toggle button 935 is set to “Month”, which displays the calendar 936 and the graphic 938 as shown in the user interface 910G.

### Exemplary Environment

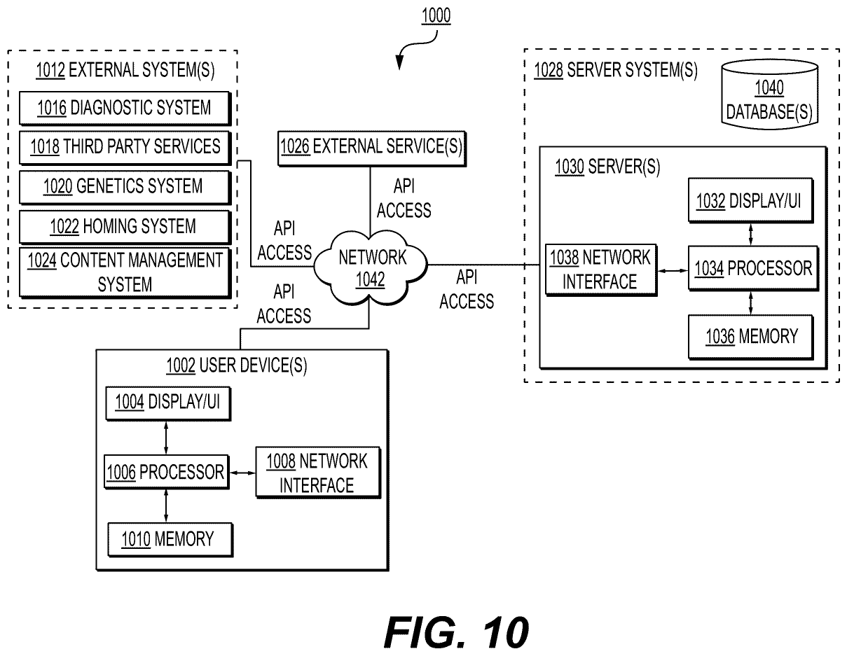

FIG. 10 depicts an exemplary environment 1000 that may be utilized with the techniques presented herein (e.g., methods 300, 320, and 340 and/or implementations of user interfaces 410A-910G). One or more user device(s) 1002, one or more external service(s) 1026, and one or more server system(s) 1028 may communicate across a network 1042. As will be discussed in further detail below, one or more server system(s) 1028 may communicate with one or more of the other components of the environment 1000 across network 1042. The one or more user device(s) 1002 may be associated with a user, e.g., a user associated with at least one pet. The systems and devices of the environment 1000 may communicate in any arrangement. As will be discussed herein, systems and/or devices of the environment 1000 may communicate in order to receive, send, and/or store data.

The user device 1002 may be configured to enable the user to access and/or interact with other systems in the environment 1000. For example, the user device 1002 may be a computer system such as, for example, a desktop computer, a mobile device, a tablet, etc. In some embodiments, the user device 1002 may include one or more electronic application(s), e.g., a program, plugin, browser extension, etc., installed on a memory of the user device 1002.

The user device 1002 may include a display/user interface (UI) 1004, a processor 1006, a memory 1010, and/or a network interface 1008. The user device 1002 may execute, by the processor 1006, an operating system (O/S) and at least one electronic application (each stored in memory 1010). The electronic application may be a desktop program, a browser program, a web client, or a mobile application program (which may also be a browser program in a mobile O/S), an applicant specific program, system control software, system monitoring software, software development tools, or the like. For example, environment 1000 may extend information on a web client that may be accessed through a web browser. In some embodiments, the electronic application(s) may be associated with one or more of the other components in the environment 1000. The application may manage the memory 1010, such as a database, to transmit streaming data to network 1042. The display/UI 1004 may be a touch screen or a display with other input systems (e.g., mouse, keyboard, etc.) so that the user(s) may interact with the application and/or the O/S. The network interface 1008 may be a TCP/IP network interface for, e.g., Ethernet or wireless communications with the network 1042. The processor 1006, while executing the application, may generate data and/or receive user inputs from the display/UI 1004 and/or receive/transmit messages to the server system(s) 1028, and may further perform one or more operations prior to providing an output to the network 1042.

External system(s) 1012 may be, for example, one or more systems that collect, manage, and/or store data corresponding to one or more pets and/or one or more pet owners. The one or more external systems may include at least one of a diagnostic system 1016 (e.g. the diagnostic system 142 of FIG. 1), a third party services system 1018 (e.g. the third party services system 182 of FIG. 1), a genetics system 1020 (e.g. the genetics system 170 of FIG. 1), a homing system 1022 (e.g. the homing system 152 of FIG. 1), and/or a content management system 1024 (e.g. the content management system 164 of FIG. 1). External system(s) 1012 may be in communication with other device(s) or system(s) in the environment 1000 over the one or more networks 1042. For example, external system(s) 1012 may communicate with the server system(s) 1028 via API (application programming interface) access over the one or more networks 1042, and also communicate with the user device(s) 1002 via web browser access over the one or more networks 1042.

External service(s) 1026 may be, for example, one or more third party and/or auxiliary systems (such as external services 122, 150, 162, 180, 190 of FIG. 1) that integrate and/or communicate with the server system(s) 1028 in performing various document information extraction tasks. External service(s) 1026 may be in communication with other device(s) or system(s) in the environment 1000 over the one or more networks 1042. For example, external service(s) 1026 may communicate with the server system(s) 1028 via API access over the one or more networks 1042, and also communicate with the user device(s) 1002 via web browser access over the one or more networks 1042.

In various embodiments, the network 1042 may be a wide area network (“WAN”), a local area network (“LAN”), a personal area network (“PAN”), or the like. In some embodiments, network 1042 may include the Internet, and information and data provided between various systems occurs online. “Online” may mean connecting to or accessing source data or information from a location remote from other devices or networks coupled to the Internet. Alternatively, “online” may refer to connecting or accessing a network (wired or wireless) via a mobile communications network or device. The Internet is a worldwide system of computer networks-a network of networks in which a party at one computer or other device connected to the network can obtain information from any other computer and communicate with parties of other computers or devices. The most widely used part of the Internet is the World Wide Web (often-abbreviated “WWW” or called “the Web”). A “website page” generally encompasses a location, data store, or the like that is, for example, hosted and/or operated by a computer system so as to be accessible online, and that may include data configured to cause a program such as a web browser to perform operations such as send, receive, or process data, generate a visual display and/or an interactive interface, or the like.

The server system(s) 1028 may include an electronic data system, e.g., a computer-readable memory such as a hard drive, flash drive, disk, etc. In some embodiments, the server system(s) 1028 includes and/or interacts with an application programming interface for exchanging data to other systems, e.g., one or more of the other components of the environment. In some examples, the server system(s) 1028 may include a server system associated with the platform 102 and/or a server system associated with the wellness system 124.

The server system(s) 1028 may include a database(s) 1040 and server(s) 1030. The server system(s) 1028 may be a computer, system of computers (e.g., rack server(s)), and/or or a cloud service computer system. The server system may store or have access to database(s) 1040 (e.g., hosted on a third party server or in memory 1036). The server(s) may include a display/UI 1032, a processor 1034, a memory 1036, and/or a network interface 1038. The display/UI 1032 may be a touch screen or a display with other input systems (e.g., mouse, keyboard, etc.) for an operator of the server(s) 1030 to control the functions of the server(s) 1030. The server system(s) 1028 may execute, by the processor 1034, an operating system (O/S) and at least one instance of a servlet program (each stored in memory 1036).

Although depicted as separate components in FIG. 10, it should be understood that a component or portion of a component in the environment 1000 may, in some embodiments, be integrated with or incorporated into one or more other components. For example, a portion of the display/UI 1032 may be integrated into the user device 1002 or the like. In some embodiments, operations or aspects of one or more of the components discussed above may be distributed amongst one or more other components. Any suitable arrangement and/or integration of the various systems and devices of the environment 1000 may be used.

In general, any process or operation discussed in this disclosure that is understood to be computer-implementable, such as the methods 300, 320, 340 illustrated in FIGS. 3A-3C, may be performed by one or more processors of a computer system, such any of the systems or devices in the environment 1000 of FIG. 10, as described above. A process or process step performed by one or more processors may also be referred to as an operation. The one or more processors may be configured to perform such processes by having access to instructions (e.g., software or computer-readable code) that, when executed by the one or more processors, cause the one or more processors to perform the processes. The instructions may be stored in a memory of the computer system. A processor may be a central processing unit (CPU), a graphics processing unit (GPU), or any suitable types of processing unit.

A computer system, such as a system or device implementing a process or operation in the examples above, may include one or more computing devices, such as one or more of the systems or devices in FIG. 10. One or more processors of a computer system may be included in a single computing device or distributed among a plurality of computing devices. A memory of the computer system may include the respective memory of each computing device of the plurality of computing devices.

### Exemplary Device

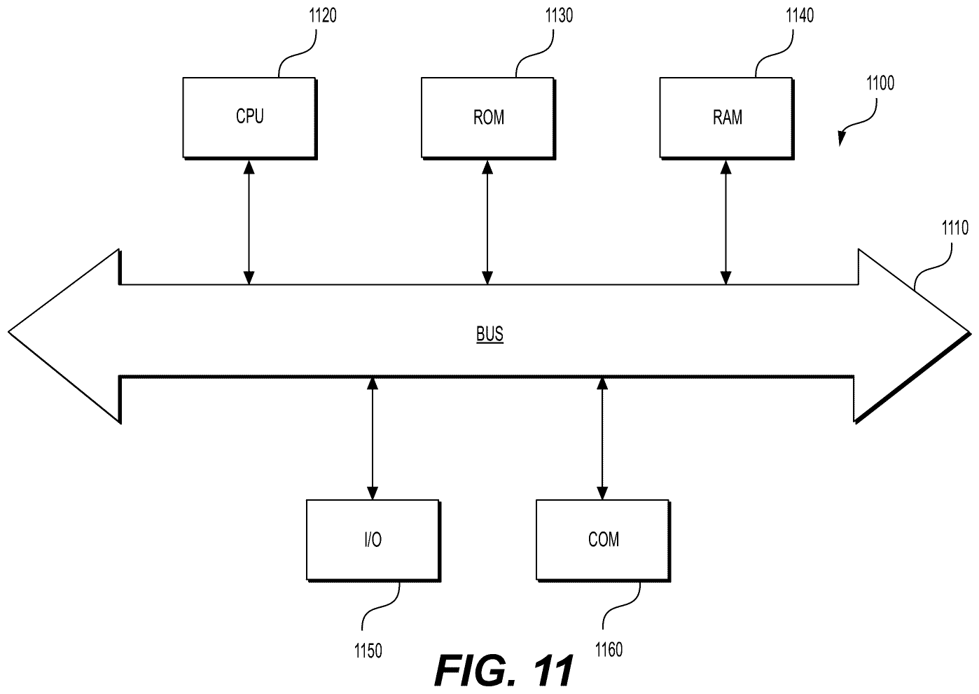

FIG. 11 is a simplified functional block diagram of a computer that may be configured as a device 1100 for executing the methods and/or implementations of FIGS. 3A-9G, according to exemplary embodiments of the present disclosure. For example, device 1100 may include a central processing unit (CPU) 1120. CPU 1120 may be any type of processor device including, for example, any type of special purpose or a general-purpose microprocessor device. As will be appreciated by persons skilled in the relevant art, CPU 1120 also may be a single processor in a multi-core/multiprocessor system, such system operating alone, or in a cluster of computing devices operating in a cluster or server farm. CPU 1120 may be connected to a data communication infrastructure 1110, for example, a bus, message queue, network, or multi-core message-passing scheme.

Device 1100 also may include a main memory 1140, for example, random access memory (RAM), and also may include a secondary memory 1130. Secondary memory 1130, e.g., a read-only memory (ROM), may be, for example, a hard disk drive or a removable storage drive. Such a removable storage drive may comprise, for example, a floppy disk drive, a magnetic tape drive, an optical disk drive, a flash memory, or the like. The removable storage drive in this example reads from and/or writes to a removable storage unit in a well-known manner. The removable storage unit may comprise a floppy disk, magnetic tape, optical disk, etc., which is read by and written to by the removable storage drive. As will be appreciated by persons skilled in the relevant art, such a removable storage unit generally includes a computer usable storage medium having stored therein computer software and/or data.

In alternative implementations, secondary memory 1130 may include other similar means for allowing computer programs or other instructions to be loaded into device 1100. Examples of such means may include a program cartridge and cartridge interface (such as that found in video game devices), a removable memory chip (such as an EPROM, or PROM) and associated socket, and other removable storage units and interfaces, which allow software and data to be transferred from a removable storage unit to device 1100.

Device 1100 also may include a communications interface (“COM”) 1160. Communications interface 1160 allows software and data to be transferred between device 1100 and external devices. Communications interface 1160 may include a modem, a network interface (such as an Ethernet card), a communications port, a PCMCIA slot and card, or the like. Software and data transferred via communications interface 1160 may be in the form of signals, which may be electronic, electromagnetic, optical, or other signals capable of being received by communications interface 1160. These signals may be provided to communications interface 1160 via a communications path of device 1100, which may be implemented using, for example, wire or cable, fiber optics, a phone line, a cellular phone link, an RF link or other communications channels.

The hardware elements, operating systems and programming languages of such equipment are conventional in nature, and it is presumed that those skilled in the art are adequately familiar therewith. Device 1100 also may include input and output ports 1150 to connect with input and output devices such as keyboards, mice, touchscreens, monitors, displays, readable media (e.g., barcode or QR code) scanner, etc. Of course, the various server functions may be implemented in a distributed fashion on a number of similar platforms, to distribute the processing load. Alternatively, the servers may be implemented by appropriate programming of one computer hardware platform.

Program aspects of the technology may be thought of as “products” or “articles of manufacture” typically in the form of executable code and/or associated data that is carried on or embodied in a type of machine-readable medium. “Storage” type media include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer of the mobile communication network into the computer platform of a server and/or from a server to the mobile device. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links, or the like, also may be considered as media bearing the software. As used herein, unless restricted to non-transitory, tangible “storage” media, terms such as computer or machine “readable medium” refer to any medium that participates in providing instructions to a processor for execution.

A computer system, such as a system or device implementing a process or operation in the examples above, may include one or more computing devices. One or more processors of a computer system may be included in a single computing device or distributed among a plurality of computing devices. A memory of the computer system may include the respective memory of each computing device of the plurality of computing devices.

A computer may be configured as a device for executing the exemplary embodiments of the present disclosure. For example, the computer may be configured according to exemplary embodiments of this disclosure. In various embodiments, any of the systems herein may be a computer including, for example, a data communication interface for packet data communication. The computer also may include a central processing unit (“CPU”), in the form of one or more processors, for executing program instructions. The computer may include an internal communication bus, and a storage unit (such as ROM, HDD, SDD, etc.) that may store data on a computer readable medium, although the computer may receive programming and data via network communications. The computer may also have a memory (such as RAM) storing instructions for executing techniques presented herein, although the instructions may be stored temporarily or permanently within other modules of computer (e.g., processor and/or computer readable medium). The computer also may include input and output ports and/or a display to connect with input and output devices such as keyboards, mice, touchscreens, monitors, displays, etc. The various system functions may be implemented in a distributed fashion on a number of similar platforms, to distribute the processing load. Alternatively, the systems may be implemented by appropriate programming of one computer hardware platform.

Furthermore, while some embodiments described herein include some but not other features included in other embodiments, combinations of features of different embodiments are meant to be within the scope of the invention, and form different embodiments, as would be understood by those skilled in the art.

Thus, while certain embodiments have been described, those skilled in the art will recognize that other and further modifications may be made thereto without departing from the spirit of the invention, and it is intended to claim all such changes and modifications as falling within the scope of the invention. For example, functionality may be added or deleted from the block diagrams and operations may be interchanged among functional blocks.

The above disclosed subject matter is to be considered illustrative, and not restrictive, and the appended claims are intended to cover all such modifications, enhancements, and other implementations, which fall within the true spirit and scope of the present disclosure. Thus, to the maximum extent allowed by law, the scope of the present disclosure is to be determined by the broadest permissible interpretation of the following claims and their equivalents, and shall not be restricted or limited by the foregoing detailed description. While various implementations of the disclosure have been described, it will be apparent to those of ordinary skill in the art that many more implementations are possible within the scope of the disclosure. Accordingly, the disclosure is not to be restricted except in light of the attached claims and their equivalents.

What is claimed is:

1. A method for annotating pet health-related sensor data, the method comprising:
receiving, by at least one processor from one or more sensors, activity data indicative of one or more movements of a pet;
determining, by the at least one processor, a behavior of the pet based on the activity data;
receiving, by the at least one processor, contextual data associated with the pet from a user device;
determining, by the at least one processor, a temporal relationship between at least one data point of the behavior of the pet and at least one data point of the contextual data;
determining, by the at least one processor, a correlation between the behavior of the pet and at least one data point of the contextual data based on the temporal relationship; and
providing, by the at least one processor, a user interface for display on the user device, wherein the user interface includes at least one graphic depicting the behavior annotated with the contextual data determined to be correlated with the behavior.

2. The method of claim 1, wherein the contextual data comprises at least one of:
a nutrition regimen of the pet;
a medication regimen of the pet;
a mobility state of the pet;
a life event of the pet; and
an emotional state of the pet.

3. The method of claim 1, further comprising:
identifying, by the at least one processor, a change in the behavior based on the activity data,
wherein determining the correlation between the behavior and the at least one data point of the contextual data comprises predicting a potential cause of the change in behavior based on the temporal relationship.

4. The method of claim 3, further comprising:
transmitting, by the at least one processor to the user device, a prompt requesting input of the contextual data in response to identifying the change in the behavior.

5. The method of claim 4, wherein the prompt comprises a plurality of selectable options, wherein each of the plurality of selectable options corresponds to a life event of the pet.

6. The method of claim 1, wherein the contextual data includes mood data, the mood data including an emotional state of the pet and a tag indicating a justification for the emotional state.

7. The method of claim 1, wherein the at least one graphic comprises a graph or plot of the behavior of the pet over a plurality of data points overlaid with at least one annotation of a life event of the pet corresponding to the at least one data point of the contextual data determined to be correlated with the behavior.

8. The method of claim 1, further comprising:
providing, by the at least one processor, a second user interface for display on the user device, wherein the second user interface includes at least one of:
a digital journal including mood data received from the user device;
a calendar indicating one or more days on which mood data was received from the user device; and
a chart indicating a frequency at which mood data was received from the user device.

9. A computer system for annotating pet health-related sensor data, the system comprising:
at least one memory having processor-readable instructions stored therein; and
at least one processor configured to access the at least one memory and execute the processor-readable instructions, which when executed by the at least one processor cause the at least one processor to perform a plurality of functions, including functions for:
receiving, from one or more sensors, activity data indicative of one or more movements of a pet;
determining a behavior of the pet based on the activity data;
receiving contextual data associated with the pet from a user device;
determining a temporal relationship between at least one data point of the behavior of the pet and at least one data point of the contextual data;
determining a correlation between the behavior of the pet and at least one data point of the contextual data based on the temporal relationship; and
providing a user interface for display on the user device, wherein the user interface includes at least one graphic depicting the behavior annotated with the contextual data determined to be correlated with the behavior.

10. The system of claim 9, wherein the contextual data comprises at least one of:
a nutrition regimen of the pet;
a medication regimen of the pet;
a mobility state of the pet;
a life event of the pet; and
an emotional state of the pet.

11. The system of claim 9, wherein the plurality of functions further includes a function for:
identifying a change in the behavior based on the activity data,
wherein determining the correlation between the behavior and the at least one data point of the contextual data comprises predicting a potential cause of the change in behavior based on the temporal relationship.

12. The system of claim 11, wherein the plurality of functions further includes a function for:
transmitting a prompt requesting input of the contextual data in response to identifying the change in the behavior.

13. The system of claim 9, wherein the contextual data includes mood data, the mood data including an emotional state of the pet and a tag indicating a justification for the emotional state.

14. The system of claim 9, wherein the at least one graphic comprises a graph or plot of the behavior of the pet over a plurality of data points overlaid with at least one annotation of a life event of the pet corresponding to the at least one data point of the contextual data determined to be correlated with the behavior.

15. The system of claim 9, wherein the plurality of functions further includes a function for:
providing a second user interface for display on the user device, wherein the second user interface includes at least one of:
a digital journal including mood data received from the user device;
a calendar indicating one or more days on which mood data was received from the user device; and
a chart indicating a frequency at which mood data was received from the user device.

16. A non-transitory computer-readable medium configured to store instructions that, when executed by at least one processor of a device for annotating pet health-related sensor data, cause the at least one processor to perform operations comprising:
receiving, from one or more sensors, activity data indicative of one or more movements of a pet;
determining a behavior of the pet based on the activity data;
receiving contextual data associated with the pet from a user device;
determining a temporal relationship between at least one data point of the behavior of the pet and at least one data point of the contextual data;
determining a correlation between the behavior of the pet and at least one data point of the contextual data based on the temporal relationship; and
providing a user interface for display on the user device, wherein the user interface includes at least one graphic depicting the behavior annotated with the contextual data determined to be correlated with the behavior.

17. The non-transitory computer-readable medium of claim 16, wherein the contextual data comprises at least one of:
a nutrition regimen of the pet;
a medication regimen of the pet;
a mobility state of the pet;
a life event of the pet; and
an emotional state of the pet.

18. The non-transitory computer-readable medium of claim 16, wherein the operations further comprise:
identifying a change in the behavior based on the activity data,
wherein determining the correlation between the behavior and the at least one data point of the contextual data comprises predicting a potential cause of the change in behavior based on the temporal relationship.

19. The non-transitory computer-readable medium of claim 18, wherein the operations further comprise:
transmitting, by the at least one processor to the user device, a prompt requesting input of the contextual data in response to identifying the change in the behavior.

20. The non-transitory computer-readable medium of claim 16, wherein the operations further comprise:
providing a second user interface for display on the user device, wherein the second user interface includes at least one of:
a digital journal including mood data received from the user device;
a calendar indicating one or more days on which mood data was received from the user device; and
a chart indicating a frequency at which mood data was received from the user device.
